(19) United States
(12) Patent Application Publication
(10) Pub. No.: US 2025/0266159 A1
(43) Pub. Date: Aug. 21, 2025

(54) SYSTEMS AND METHODS FOR PERFORMING MEDICAL TASKS USING A MEDICAL ARTIFICIAL INTELLIGENCE SYSTEM

(71) Applicant: President and Fellows of Harvard College, Cambridge, MA (US)

(72) Inventors: Pranav Rajpurkar, Cambridge, MA (US); Hong-Yu Zhou, Cambridge, MA (US)

(73) Assignee: President and Fellows of Harvard College, Cambridge, MA (US)

(21) Appl. No.: 19/057,629

(22) Filed: Feb. 19, 2025

Related U.S. Application Data
(60) Provisional application No. 63/647,326, filed on May 14, 2024.
(60) Provisional application No. 63/555,589, filed on Feb. 20, 2024.

(51) Int. Cl. G16H 40/20; G06F 40/284; G06T 7/00; G16H 15/00; G16H 30/40

(52) U.S. Cl. CPC G16H 40/20; G06F 40/284; G06T 7/0012; G16H 15/00; G16H 30/40; G06T 2207/30004

(57) ABSTRACT

Techniques for performing medical tasks using a medical artificial intelligence (MAI) system including receiving multi-modal input; processing at least a portion of the multi-modal input using a trained LLM to obtain LLM output indicating that zero, one, or multiple tasks are to be additionally performed; when the LLM output indicates that zero tasks are to be additionally performed, outputting at least some of the LLM output as a response to the request; and when the LLM output indicates that one or multiple tasks are to be additionally performed, processing at least some of the multi-modal input, using at least one of a plurality of task-specific software tools and the LLM output, to obtain at least one task-specific output; and outputting a response generated using the at least some of the LLM output and the at least one task-specific output as a response to the request.

### CROSS-REFERENCE TO RELATED APPLICATIONS

This application claims the benefit of priority under 35 U.S.C. § 119(e) to U.S. Provisional Patent Application No. 63/555,589, filed Feb. 20, 2024 titled “SYSTEMS AND METHODS FOR PERFORMING MEDICAL TASKS USING A MEDICAL ARTIFICIAL INTELLIGENCE SYSTEM” under Attorney Docket No.: H0776.70169US00, and to U.S. Provisional Patent Application No. 63/647,326, Filed May 14, 2024 titled “SYSTEMS AND METHODS FOR PERFORMING MEDICAL TASKS USING A MEDICAL ARTIFICIAL INTELLIGENCE SYSTEM” under Attorney Docket No.: H0776.70169US01, each of which is hereby incorporated by reference in its entirety herein.

### BACKGROUND

The use of artificial intelligence (AI) to help perform medical tasks has been developed over recent years. Use of such medical artificial intelligence (MAI) has enabled advances on performance of certain specific medical tasks, enabling increased diagnostic precision and patient care.

### SUMMARY

According to some aspects, there is provided a method for performing medical tasks using a medical artificial intelligence (MAI) system, the MAI system comprising a trained large language model (LLM) and a plurality of task-specific software tools, the method comprising: using at least one computer hardware processor to perform: (A) receiving multi-modal input comprising image input and a request that the MAI system perform at least one medical task on the multi-modal input; (B) processing at least a portion of the multi-modal input using the trained LLM to obtain LLM output, the LLM output indicating that zero, one, or multiple tasks are to be additionally performed by at least one of the plurality of task-specific software tools; (C) when the LLM output indicates that zero tasks are to be additionally performed, outputting at least some of the LLM output as a response to the request; and (D) when the LLM output indicates that one or multiple tasks are to be additionally performed, processing at least some of the multi-modal input, using the at least one of the plurality of task-specific software tools and the LLM output, to obtain at least one task-specific output; outputting a response generated using the at least some of the LLM output and the at least one task-specific output as a response to the request.

According to some aspects, there is provided a method for performing medical tasks using a medical artificial intelligence (MAI) system, the MAI system comprising a plurality of modules including a multi-modal input coordinator module, an orchestrator module comprising a trained large language model (LLM), and a plurality of task-specific software tools, the method comprising: executing the multi-modal input coordinator module, using at least one computer hardware processor, to perform: (A) receiving multi-modal input comprising image input and text input indicating a request that the MAI system perform at least one medical task on the multi-modal input; (B) processing the multi-modal input to obtain a tokenized representation of the image input and the text input; and executing the orchestrator module, using the at least one computer hardware processor, to perform: (C) processing the tokenized representation using the trained LLM to obtain LLM output at least partially responsive to the request, the LLM output comprising latent embeddings and textual output, the LLM output indicating zero, one, or multiple tasks are to be additionally performed by at least one of the plurality of task-specific software tools; (D) when the LLM output indicates that zero tasks are to be additionally performed by the at least one of the plurality of task-specific software tools, outputting the textual output as a response to the request of the MAI system; and (E) when the LLM output indicates that one or multiple tasks are to be additionally performed by the at least one of the plurality of task-specific software tools, identifying, based on the LLM output and from among the plurality of task-specific software tools, a first task-specific software tool; generating, from the latent embeddings and the multi-modal input, first input for the first task-specific software tool and processing the first input with the first task-specific software tool to obtain a first task-specific output; generating an integrated response to the request of the MAI system using the textual output produced by the trained LLM and the first task-specific output generated by the first task-specific software tool; and outputting the integrated response as a response to the request of the MAI system.

According to some aspects, there is provided a method for training a medical artificial intelligence (MAI) system to perform medical tasks, the MAI system comprising an LLM and a plurality of task-specific software tools, wherein the LLM is to be trained to process multi-modal input, containing image input and a request that the MAI system perform at least one medical task on the multi-modal input, to obtain corresponding LLM output at least partially responsive to the request and indicating that zero, one, or multiple tasks are to be additionally performed by at least one of the plurality of task-specific software tools, the method comprising: obtaining training data comprising multiple multi-modal inputs, each particular one of the multiple multi-modal inputs comprising a respective image input and a respective request that the MAI system perform at least one respective medical task on the particular multi-modal input; training the LLM using the training data to obtain a trained LLM.

According to some aspects, there is provided system comprising: at least one computer hardware processor; and at least one non-transitory computer-readable storage medium having encoded thereon instructions that, when executed by the at least one computer hardware processor, cause the at least one computer hardware processor to perform any of the methods described herein.

According to some aspects, there is provided at least one non-transitory computer-readable storage medium having encoded thereon instructions that, when executed by at least one computer hardware processor, cause the at least one computer hardware processor to perform any of the methods described herein.

### BRIEF DESCRIPTION OF THE DRAWINGS

Various aspects and embodiments will be described with reference to the following figures. It should be appreciated that the figures are not necessarily drawn to scale. For purposes of clarity, not every component may be labeled in every drawing. In the drawings:

FIG. 1 illustrates an example medical artificial intelligence (MAI) system, in accordance with some embodiments of the technology described herein.

FIG. 2 illustrates an example method for performing medical tasks using the MAI system of FIG. 1, in accordance with some embodiments of the technology described herein.

FIG. 3 illustrates components of the MAI system of FIG. 1, in accordance with some embodiments of the technology described herein.

FIG. 4 illustrates another example method for performing medical tasks using the MAI system of FIG. 1, in accordance with some embodiments of the technology described herein.

FIG. 5 illustrates an example architecture of a vision-language adapter of the MAI system of FIG. 1, in accordance with some embodiments of the technology described herein.

FIG. 6A illustrates an example of a visual detection tool, in accordance with some embodiments of the technology described herein.

FIG. 6B illustrates an example of a two-dimensional visual segmentation tool, in accordance with some embodiments of the technology described herein.

FIG. 6C illustrates an example of a three-dimensional visual segmentation tool, in accordance with some embodiments of the technology described herein.

FIG. 7A illustrates an example workflow for performing a chest pathology detection task using the MAI system of FIG. 1, in accordance with some embodiments of the technology described herein.

FIG. 7B illustrates an example workflow for performing an abdominal organ segmentation task using the MAI system of FIG. 1, in accordance with some embodiments of the technology described herein.

FIG. 7C illustrates an example workflow for performing a longitudinal study comparisons task using the MAI system of FIG. 1, in accordance with some embodiments of the technology described herein.

FIG. 8 illustrates an example method for training an MAI system to perform medical tasks, in accordance with some embodiments of the technology described herein.

FIG. 9 is an illustrative implementation of a computer system that may be used in connection with some embodiments of the technology described herein.

FIG. 10A illustrates example data showing improvement of the MAI system described herein over existing models across eleven tasks, in accordance with some embodiments of the technology described herein.

FIG. 10B illustrates aspects of the data used to train the MAI system described herein, in accordance with some embodiments of the technology described herein.

FIG. 11A illustrates example results of performance of the MAI system described herein on an anatomical structure detection task, in accordance with some embodiments of the technology described herein.

FIG. 11B illustrates example results of performance of the MAI system described herein on a chest pathology detection task, in accordance with some embodiments of the technology described herein.

FIG. 11C illustrates example results of performance of the MAI system described herein on a chest major organ segmentation task, in accordance with some embodiments of the technology described herein.

FIG. 11D illustrates example results of performance of the MAI system described herein on a skin lesion segmentation task, in accordance with some embodiments of the technology described herein.

FIG. 11E illustrates example results of performance of the MAI system described herein on an abdominal organ segmentation task, in accordance with some embodiments of the technology described herein.

FIG. 12A illustrates example results of performance of the MAI system described herein on a chest pathology classification task, in accordance with some embodiments of the technology described herein.

FIG. 12B illustrates example results of performance of the MAI system described herein on a skin lesion classification task, in accordance with some embodiments of the technology described herein.

FIG. 13 illustrates comparative analyses of learning from multimodal supervision. The impact of training MedVersa with different types of tasks, in accordance with some embodiments of the technology described herein.

FIG. 14 illustrates a table showing segmentation results of skin lesions and abdominal organs, in accordance with some embodiments of the technology described herein.

### DETAILED DESCRIPTION

Aspects of the technology described herein provides techniques for performing medical tasks using a medical artificial intelligence (MAI) system. The techniques may be embodied in one or more methods, systems, and/or non-transitory computer readable media having instructions encoded thereon.

Medical artificial intelligence (MAI) has made advances on performing medical tasks. Existing efforts have been devoted to developing solutions for specific tasks. However, the inventors have recognized that the task-specific implementation of MAI may result in narrow use cases in real-world clinical deployment.

In addition, existing MAI models have been designed to learn from natural language supervision and thus can only produce textual outputs. While this paradigm may be suitable for certain tasks (e.g., vision-language tasks), it does not readily apply to a majority of vision-centric problems such as detection and segmentation, which are indispensable to medical image interpretation. For example, it is almost impossible to describe the contour of the kidney using only words. Moreover, accurate diagnosis of conditions such as chronic obstructive pulmonary disease requires the ability to localize, segment lungs, and detect disease indicators (e.g., lung enlargement, flattened/lower diaphragm), which are tasks that may be difficult or impossible to perform using natural language alone.

To address this, the inventors have developed a generalist MAI system for more flexible problem solving that is configured to (e.g., trained to) process multi-modal input and/or generate multi-modal output. In this sense, the MAI system may be referred to as a “generalist” MAI system. In contrast to task-specific AI, the generalist MAI system developed by the inventors and described herein allows for dynamic task specification using specialized tools for varying tasks and further is capable of processing multi-modal inputs and generating multi-modal outputs.

In particular, the MAI system described herein is designed to perform a plurality of different medical tasks, including vision-language tasks and vision-centric tasks. For example, the plurality of tasks capable of being performed using the technology described herein include vision-language tasks, including by way of example, one or more of radiology report generation, longitudinal study comparison, region-of-interest captioning, open-ended visual question answering (VQA), chest pathology classification, and/or skin lesion classification, and/or one or more vision-centric tasks, including by way of example, one or more medical image analysis tasks including one or more of anatomical detection, abnormality detection and/or segmentation, chest abnormality detection and/or segmentation, pathology classification, lesion segmentation and/or organ segmentation.

The MAI system is designed to perform one or more medical tasks on multi-modal input. That is, the MAI system is configured to receive input of at least two different types. The input may comprise text input and image input. The image input may comprise one or multiple two-dimensional images, one or multiple three-dimensional images, and/or a video. In some embodiments, the image input is a single image. In some embodiments, the image input is multiple (e.g., two or more) images. The multiple images may comprise multiple views of at least a portion of a patient (e.g., which may be at approximately the same point in time). In some embodiments, the multiple images may comprise approximately the same view of at least a portion of a patient at multiple different points in time. In some embodiments, the one or more images may be one or more medical images, including one or more radiographs, dermoscopy images, and/or computed tomography scans.

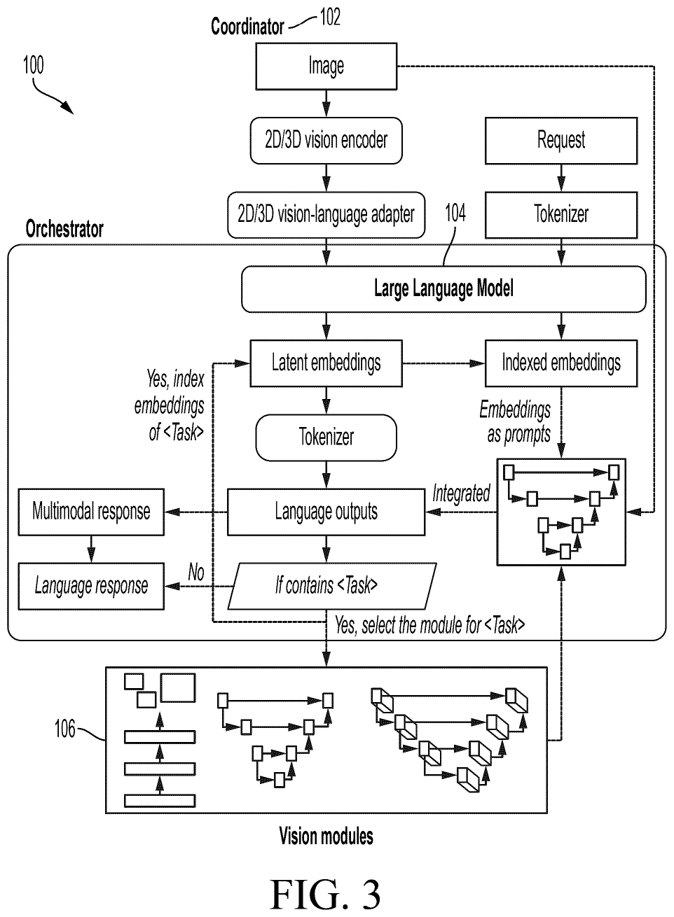

The MAI system described herein may be implemented in software and, as such, may be composed of a plurality of software modules. Each software module may include processor-executable instructions configured to perform one or more functions described herein. For example, the MAI system may comprise a multi-modal input coordinator module. The multi-modal input coordinator module may be configured to receive the multi-modal input to the MAI system and process the multi-modal input. For example, the processing of the multi-modal input by the MAI may comprise tokenizing the multi-modal input to obtain a tokenized representation of the multi-modal input, as described herein, for example with respect to FIG. 3, including in paragraphs 44 and 92-95, for example.

In some embodiments, the MAI system may further comprise an orchestrator module. The orchestrator module may include a trained large language model (LLM). The orchestrator module uses the large language model as an orchestrator for flexible input (including medical image input) interpretation. The orchestrator module may comprise and/or be configured to interact with one or multiple task-specific software tools (e.g., task-specific trained machine learning models) stored in a repository. The orchestrator module may be configured to generate a response to a request for the MAI system to perform at least one medical task. The orchestrator module may be configured to generate the response to the at least one medical task at least in part by generating an LLM output using the LLM. The orchestrator module may be further configured to determine whether to use at least one of the plurality of task-specific software tools to perform one or more additional tasks. If the orchestrator module determines to use the at least one of the plurality of task-specific software tools to perform one or more additional tasks, the at least one of the plurality of task-specific software tools may be used to generate at least one task-specific output, which may be integrated with the LLM output to produce a response to the at least one medical task. The orchestrator module is further described herein, including, with respect to FIG. 3 and in paragraphs 96-99.

The MAI system described herein produces an output as a response to a request to perform the at least one medical task. For example, the response to the request output by the MAI system may comprise one or more of a medical report, a segmented image, an indication of a classified medical condition, a comparison of longitudinal study images, an indication of a detected anatomical structure, and/or an indication of a detected abnormality.

The output may be an integrated output, which is generated based on multiple software tools. For example, the integrated output may comprise a component generated by a large language model (e.g., the LLM part of the orchestrator module) and a component generated by one or more task-specific software tools (e.g., a task-specific trained machine learning model). In some embodiments, the output comprises text alone. In some embodiments, the output comprises one or more images, alone. In some embodiments, the output of the MAI system is multi-modal. For example, in some such embodiments, the output may comprise a text and one or more images.

According to some embodiments, there is provided a method for performing medical tasks (e.g., one or more vision-language tasks comprising one or more of radiology report generation, longitudinal study comparison, region-of-interest captioning, open-ended visual question answering (VQA), chest pathology classification, and/or skin lesion classification, and/or one or more vision-centric tasks comprising one or more medical image analysis tasks including one or more of anatomical detection, chest abnormality detection, pathology classification, lesion segmentation and/or organ segmentation) using a medical artificial intelligence (MAI) system, the MAI system comprising a trained large language model (LLM) and a plurality of task-specific software tools (e.g., one or more task-specific trained machine learning models), the method comprising: using at least one computer hardware processor to perform: (A) receiving multi-modal input comprising image input (e.g., one or more medical images such as one or more radiographs, dermoscopy images, and/or computed tomography scans, a single image, multiple images such as multiple views of at least a portion of a patient and/or a same view of at least a portion of a patient at multiple points in time, one or more two-dimensional images, one or more three-dimensional images, one or more videos) and a request that the MAI system perform at least one medical task on the multi-modal input; (B) processing at least a portion of the multi-modal input using the trained LLM to obtain LLM output, the LLM output indicating that zero, one, or multiple tasks are to be additionally performed by at least one of the plurality of task-specific software tools (e.g., at least one task-specific trained machine learning model); (C) when the LLM output indicates that zero tasks are to be additionally performed, outputting at least some of the LLM output as a response to the request; and (D) when the LLM output indicates that one or multiple tasks are to be additionally performed, processing at least some of the multi-modal input, using the at least one of the plurality of task-specific software tools and the LLM output, to obtain at least one task-specific output; outputting a response (e.g., multi-modal output such as a text output and an image output; one or more of a medical report, a segmented image, an indication of a classified medical condition, a comparison of longitudinal study images, an indication of a detected anatomical structure and/or an indication of a detected abnormality) generated using the at least some of the LLM output and the at least one task-specific output as a response to the request. In some embodiments, the LLM output comprises a text output and the at least one task-specific output comprises an image output.

In some embodiments, at least a portion of the multi-modal input includes an output previously obtained from the MAI system. In this way, a user may interact with the MAI system to perform multiple task. The user may provide a multi-modal input to the MAI system for processing and receive a corresponding output. The user may review the corresponding output and then provide a second multi-modal input based on the output (e.g., the second multi-modal input may include at least a portion of, for example an image in, the output) to the MAI system for further processing. The second multi-modal input may thus include at least a part of the output previously provided by the MAI and/or additional input provided by the user. Such interactions may continue until the MAI system performs all the tasks the user wishes that the MAI system to perform.

In some embodiments, the response comprises a multi-modal output, which may comprise a text output and an image output. In some embodiments, the LLM output comprises the text output and the at least one task-specific output comprises the image output.

In some embodiments, the method further comprises processing the multi-modal input to obtain a tokenized representation of the image input and the text input at least in part by processing the text input to generate text tokens and processing the image input to generate visual tokens, and wherein the at least a portion of the multi-modal output comprises the tokenized representation of the image input and the text input (e.g., by adapting the visual tokens into text tokens using a model trained to transform visual tokens into text tokens). In some embodiments, when the image input comprises a two-dimensional (2D) image, the processing the image input comprises processing the 2D image using a 2D vision encoder; and when the image input comprises a three-dimensional (3D) image, the processing the image input comprises processing the 3D image using a 3D vision encoder.

In some embodiments, the method further comprises determining whether the LLM output indicates zero, one, or multiple tasks are to be additionally performed by the at least one of the plurality of task-specific software tools (e.g., by determining whether the LLM output comprises at least one tag associated with at least one respective task). In some embodiments, the method further comprises identifying the at least one of the plurality of task-specific software tools using the at least one tag, wherein the at least one tag comprises a first tag associated with a first task, and the at least one of the plurality of task-specific software tools is trained to perform the first task.

In some embodiments, the image input comprises a two-dimensional image; the LLM output indicates that a detection task is to be additionally performed by the at least one of the plurality of task-specific software tools; the at least one of the plurality of task-specific software tools comprises a task-specific software tool trained to perform the detection task on the two-dimensional image; the at least one task-specific output comprises a second textual output, wherein obtaining the at least one task-specific output comprises generating the second textual output; and the response comprises the LLM output and the second textual output, wherein outputting the response comprises outputting the LLM output and the second textual output.

In some embodiments, the image input comprises a three-dimensional image; the LLM output indicates that a segmentation task is to be additionally performed by the at least one of the plurality of task-specific software tools; the at least one of the plurality of task-specific software tools comprises a task-specific software tool trained to perform the segmentation task on the three-dimensional image; the at least one task-specific output comprises an image output, wherein obtaining the at least one task-specific output comprises generating the image output; and the response comprises the image output and the LLM output, wherein outputting the response comprises outputting the LLM output and the image output.

In some embodiments, the image input comprises a plurality of two-dimensional images; and the LLM output indicates that zero additional tasks are to be additionally performed by the at least one of the plurality of task-specific software tools.

In some embodiments, the method further comprises indexing latent embeddings of task specification tokens corresponding to the tasks to be additionally performed by the at least one of the plurality of task-specific software tools and encoding the indexed latent embeddings into a repository comprising the plurality of task-specific software tools.

In some embodiments, the method further comprises updating a repository storing the plurality of task-specific software tools, wherein updating the repository comprises adjusting how one or more of the plurality of task-specific software tools performs a task and/or adding one or more additional task-specific software tools to the repository.

In some embodiments, the method further comprises training the trained LLM using a plurality of tags, each tag of the plurality of tags is associated a respective task of the tasks to be additionally performed by the at least one of the plurality of task-specific software tools. In some embodiments, the method further comprises optimizing the LLM using domain-aware minibatch gradient descent (e.g., to create homogeneous minibatches for training, each of the homogenous minibatches specific to a single type of task of tasks to be additionally performed by the at least one of the plurality of task-specific software tools and a single type of imaging modality).

According to some embodiments, there is provided a method for performing medical tasks (e.g., one or more vision-language tasks comprising one or more of radiology report generation, longitudinal study comparison, region-of-interest captioning, open-ended visual question answering (VQA), chest pathology classification, and/or skin lesion classification, and/or one or more vision-centric tasks comprising one or more medical image analysis tasks including one or more of anatomical detection, chest abnormality detection, pathology classification, lesion segmentation and/or organ segmentation) using a medical artificial intelligence (MAI) system, the MAI system comprising a plurality of modules including a multi-modal input coordinator module, an orchestrator module comprising a trained large language model (LLM), and a plurality of task-specific software tools (e.g., one or more task-specific trained machine learning models), the method comprising: executing the multi-modal input coordinator module, using at least one computer hardware processor, to perform: (A) receiving multi-modal input comprising image input (e.g., one or more medical images such as one or more radiographs, dermoscopy images, and/or computed tomography scans, a single image, multiple images such as multiple views of at least a portion of a patient and/or a same view of at least a portion of a patient at multiple points in time, one or more two-dimensional images, one or more three-dimensional images, one or more videos) and text input indicating a request that the MAI system perform at least one medical task on the multi-modal input; (B) processing the multi-modal input to obtain a tokenized representation of the image input and the text input; and executing the orchestrator module, using the at least one computer hardware processor, to perform: (C) processing the tokenized representation using the trained LLM to obtain LLM output at least partially responsive to the request, the LLM output comprising latent embeddings and textual output, the LLM output indicating zero, one, or multiple tasks are to be additionally performed by at least one of the plurality of task-specific software tools (e.g., at least one task-specific trained machine learning model); (D) when the LLM output indicates that zero tasks are to be additionally performed by the at least one of the plurality of task-specific software tools, outputting the textual output as a response to the request of the MAI system; and (E) when the LLM output indicates that one or multiple tasks are to be additionally performed by the at least one of the plurality of task-specific software tools, identifying, based on the LLM output and from among the plurality of task-specific software tools, a first task-specific software tool; generating, from the latent embeddings and the multi-modal input, first input for the first task-specific software tool and processing the first input with the first task-specific software tool to obtain a first task-specific output; generating an integrated response (e.g., multi-modal output such as a text output and an image output; one or more of a medical report, a segmented image, an indication of a classified medical condition, a comparison of longitudinal study images, an indication of a detected anatomical structure and/or an indication of a detected abnormality) to the request of the MAI system using the textual output produced by the trained LLM and the first task-specific output generated by the first task-specific software tool; and outputting the integrated response as a response to the request of the MAI system.

In some embodiments, at least a portion of the multi-modal input includes an output previously obtained from the MAI system. In this way, a user may interact with the MAI system to perform multiple task. The user may provide a multi-modal input to the MAI system for processing and receive a corresponding output. The user may review the corresponding output and then provide a second multi-modal input based on the output (e.g., the second multi-modal input may include at least a portion of, for example an image in, the output) to the MAI system for further processing. The second multi-modal input may thus include at least a part of the output previously provided by the MAI and/or additional input provided by the user. Such interactions may continue until the MAI system performs all the tasks the user wishes that the MAI system to perform.

In some embodiments, the response comprises a multi-modal output, which may comprise a text output and an image output. In some embodiments, the LLM output comprises the text output and the at least one task-specific output comprises the image output.

In some embodiments, processing the multi-modal input to obtain the tokenized representation of the image input and the text input comprises processing the text input to generate text tokens and processing the image input to generate visual tokens (e.g., by adapting the visual tokens into text tokens using a model trained to transform visual tokens into text tokens). In some embodiments, when the image input comprises a two-dimensional (2D) image, the processing the image input comprises processing the 2D image using a 2D vision encoder; and when the image input comprises a three-dimensional (3D) image, the processing the image input comprises processing the 3D image using a 3D vision encoder.

In some embodiments, the method further comprises determining whether the LLM output indicates zero, one, or multiple tasks are to be additionally performed by the at least one of the plurality of task-specific software tools (e.g., by determining whether the LLM output comprises at least one tag associated with at least one respective task). In some embodiments, the method further comprises identifying the at least one of the plurality of task-specific software tools using the at least one tag, wherein the at least one tag comprises a first tag associated with a first task, and the at least one of the plurality of task-specific software tools is trained to perform the first task.

In some embodiments, the image input comprises a two-dimensional image; the LLM output indicates that a detection task is to be additionally performed by the at least one of the plurality of task-specific software tools; the first task-specific software tool comprises a task-specific software tool trained to perform the detection task on the two-dimensional image; the first task-specific output comprises a second textual output, wherein generating the first task-specific output comprises generating the second textual output; and the integrated response comprises the textual output of the LLM output and the second textual output, wherein outputting the integrated response comprises outputting the textual output of the LLM output and the second textual output.

In some embodiments, the image input comprises a three-dimensional image; the LLM output indicates that a segmentation task is to be additionally performed by the at least one of the plurality of task-specific software tools; the first task-specific software tool comprises a task-specific software tool trained to perform the segmentation task on the three-dimensional image; the first task-specific output comprises an image output, wherein generating the first task-specific output comprises generating the image output; and the integrated response comprises the image output and the textual output of the LLM output, wherein outputting the integrated response comprises outputting the textual output of the LLM output and the image output.

In some embodiments, the image input comprises a plurality of two-dimensional images; and the LLM output indicates that zero additional tasks are to be additionally performed by the at least one of the plurality of task-specific software tools.

In some embodiments, the method further comprises indexing latent embeddings of task specification tokens corresponding to the tasks to be additionally performed by the at least one of the plurality of task-specific software tools and encoding the indexed latent embeddings into a repository comprising the plurality of task-specific software tools.

In some embodiments, the method further comprises updating a repository storing the plurality of task-specific software tools, wherein updating the repository comprises adjusting how one or more of the plurality of task-specific software tools performs a task and/or adding one or more additional task-specific software tools to the repository.

In some embodiments, the method further comprises training the trained LLM using a plurality of tags, each tag of the plurality of tags is associated a respective task of the tasks to be additionally performed by the at least one of the plurality of task-specific software tools. In some embodiments, the method further comprises training an LLM, to obtain the trained LLM, using referring image instructions, which enclose the visual tokens of different images with different text identifiers and incorporating the text identifiers into text instructions to help the LLM specify different images. In some embodiments, the method further comprises optimizing the LLM using domain-aware minibatch gradient descent (e.g., to create homogeneous minibatches for training, each of the homogenous minibatches specific to a single type of task of tasks to be additionally performed by the at least one of the plurality of task-specific software tools and a single type of imaging modality).

According to some embodiments, there is provided a method for training a medical artificial intelligence (MAI) system to perform medical tasks, the MAI system comprising an LLM and a plurality of task-specific software tools, wherein the LLM is to be trained to process multi-modal input, containing image input and a request that the MAI system perform at least one medical task on the multi-modal input, to obtain corresponding LLM output at least partially responsive to the request and indicating that zero, one, or multiple tasks are to be additionally performed by at least one of the plurality of task-specific software tools, the method comprising: obtaining training data comprising multiple multi-modal inputs, each particular one of the multiple multi-modal inputs comprising a respective image input and a respective request that the MAI system perform at least one respective medical task on the particular multi-modal input; training the LLM using the training data to obtain a trained LLM. In some embodiments, the method further comprises using the trained LLM according to any of the methods for performing medical tasks using a medical artificial intelligence (MAI) system described herein. In some embodiments, the training is performed using domain-aware minibatch gradient descent.

According to some embodiments, there is provided a medical artificial intelligence (MAI) system comprising: a trained large language model (LLM); a plurality of task-specific trained software tools; at least one computer hardware processor; and at least one non-transitory computer-readable storage medium having encoded thereon instructions that, when executed by the at least one computer hardware processor, cause the at least one computer hardware processor to perform any of the methods described herein. In some embodiments, the MAI system further comprises a plurality of modules including a multi-modal input coordinator module and an orchestrator module comprising the trained LLM and the plurality of task-specific trained software tools.

According to some embodiments, there is provided at least one non-transitory computer-readable storage medium having encoded thereon instructions that, when executed by at least one computer hardware processor, cause the at least one computer hardware processor to perform any of the methods described herein.

The aspects and embodiments described above, as well as additional aspects and embodiments, are described further below. These aspects and/or embodiments may be used individually, all together, or in any combination, as the application is not limited in this respect.

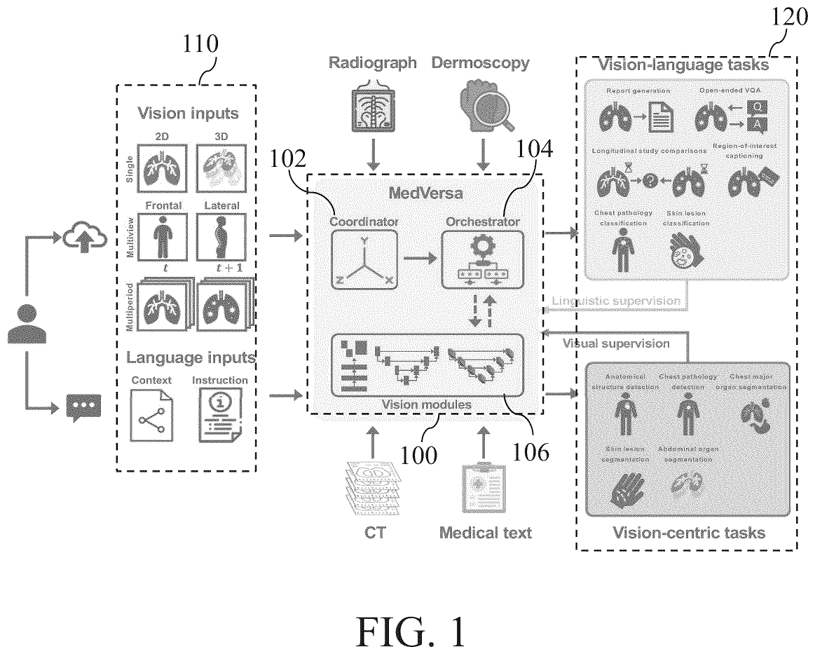

Aspects of the technology described herein relate to a medical artificial intelligence (MAI) system for performing one or more medical tasks. FIG. 1 illustrates an example MAI system, in accordance with some embodiments of the technology described herein.

The MAI system 100 is capable of performing a medical task. For example, the MAI system is configured to perform one or more vision-language tasks including one or more of medical report generation, longitudinal study comparison, region-of-interest captioning, open-ended visual question answering, and/or abnormality (e.g., skin lesion) classification. In some embodiments, the MAI system is additionally or alternatively configured to perform one or more vision-centric tasks comprising one or more medical image analysis tasks including one or more of anatomical structure identification, abnormality characterization, chest abnormality detection, lesion segmentation, and/or organ segmentation.

The MAI system is enabled to perform multiple different medical tasks, including multiple different vision-language and vision-centric tasks. The MAI system combines linguistic and visual processing capabilities which allows the MAI system to handle both vision-language and vision-centric tasks effectively. This versatility is particularly important in medica settings, where diverse data types and diagnostic requirements are common.

As described herein, the MAI system is configured to determine a medical task to before based on an input. Input to the MAI system 100 may include a variety of different inputs 110. For example, the input 110 can one or more vision input(s) and/or one or more language input(s). Accordingly, the input to the MAI system 100 may be multi-modal (e.g., including two different types of input such as vision and language inputs).

In some embodiments, the input comprises an image input. The image input may be one or more medical images. The one or more medical images may be or more radiographs, dermoscopy images, computed tomography scans, pathology images, ultrasound images, endoscopy images, and/or magnetic resonance imaging (MRI) images.

In some embodiments, the image input comprises one or more two-dimensional (2D) medical images (e.g., a single image or multiple images). In some embodiments, the image input comprises one or more three-dimensional (3D) medical images (e.g., a single image or multiple images). In some embodiments, the image input comprises one or more videos (e.g., multiple 2D and/or 3D images in a sequence).

In some embodiments, where the image input comprises multiple images, the multiple images may provide multiple views of at least a portion of a patient. For example, a first image may be a frontal view and a second image may be a lateral view. In some embodiments, the multiple images comprise a same portion of a patient at different points in time (e.g., multiperiod data from time t to time t+1).

The input 110 may comprise text input. In some embodiments, the text input comprises an instruction from which a task may be derived. In some embodiments, the text input may additionally or alternatively comprise context which the MAI system uses to perform the desired task.

It should be appreciated that the inputs may be a combination of the inputs described above. For example, the MAI system enables multi-modal inputs. As such the input may comprise image (e.g., vision) input and text (e.g., language) input.

The MAI system 100 is further configured to produce an output 120 representing a response or result of the medical task. In some embodiments, the output 120 comprises a multi-modal output. For example, as shown in FIG. 1, the output 120 may comprise text (e.g., language) output and/or image (e.g., vision) output.

The output may be generated by the orchestrator module 104 described herein. The orchestrator module 104 may use one or more tools from a specialist tool repository 106 to perform a medical task based on input received and processed by a coordinator module 102.

Examples of outputs of the MAI system include one or more of a medical report (e.g., in response to a report generation task), a captioned image (e.g., in response to a captioning task such as region-of-interest captioning), a segmented image (e.g., in response to a segmentation task such as chest major organ segmentation, skin lesion segmentation, abdominal organ segmentation), an indication of a classified medical condition (e.g., in response to a classification task such as chest pathology classification and/or skin lesion classification), a comparison of longitudinal study images (e.g., in response to a classification task), an indication of a detected anatomical structure and/or an indication of a detected abnormality (e.g., in response to a detection task such as anatomical structure detection and/or chest pathology detection). In contrast to existing models, the MAI system 100 is capable of generating a response to vision language and vision centric tasks, as shown in FIG. 1.

As shown in FIG. 1, the MAI system 100 comprises a coordinator module 102, an orchestrator module 104, and a specialist tool repository 106. As described herein, the coordinator module 102 recites the input 110. The coordinator module 102 processes the input before providing the processed input to an orchestrator module 104.

The orchestrator module 104 assesses how to perform the requested medical task based on the input. For example, as described herein, the orchestrator module 104 may comprise a large language model (LLM). The orchestrator module 104 may determine, based on the desired task, whether to perform the task using the LLM alone, or whether to additionally utilize one or more specialized tools from the specialist tool repository 106.

The orchestrator module 104 therefore dynamically coordinates with specialist tools (e.g., trained machine learning models, including trained task-specific machine learning modules) to perform a task (including vision centric tasks). The orchestrator module 104 interprets the processed input received from the coordinator module 102 to determine whether to utilize one or more of the specialized tools in the specialist tool repository to carry out the desired task. The integration of specialist tools within the MAI system 100 enhances its capability in areas where language-based models traditionally falter, such as detailed image analysis required in chest abnormality detection and skin lesion segmentation. The comprehensive approach, combining the contextual decision-making of the large language model with the precision of specialist tools, offers a more robust and versatile diagnostic tool.

As described herein, the MAI system 100 includes the specialist tool repository 106. The specialist tool repository 106 stores a plurality of task specific software tools that the LLM of the orchestrator module 104 may utilize to perform a medical task. The plurality of task-specific software tools may comprise one or more task-specific trained machine learning models.

The MAI system 100 may be updated as desired to update or add to the plurality of task-specific software tools stored in the specialist tool repository 106. For example, adding addition task-specific software tools to the repository may enable the MAI system to perform new medical tasks, thus enabling the MAI system to adapt and grow in respond to evolving medical imaging techniques and diagnostic requirements.

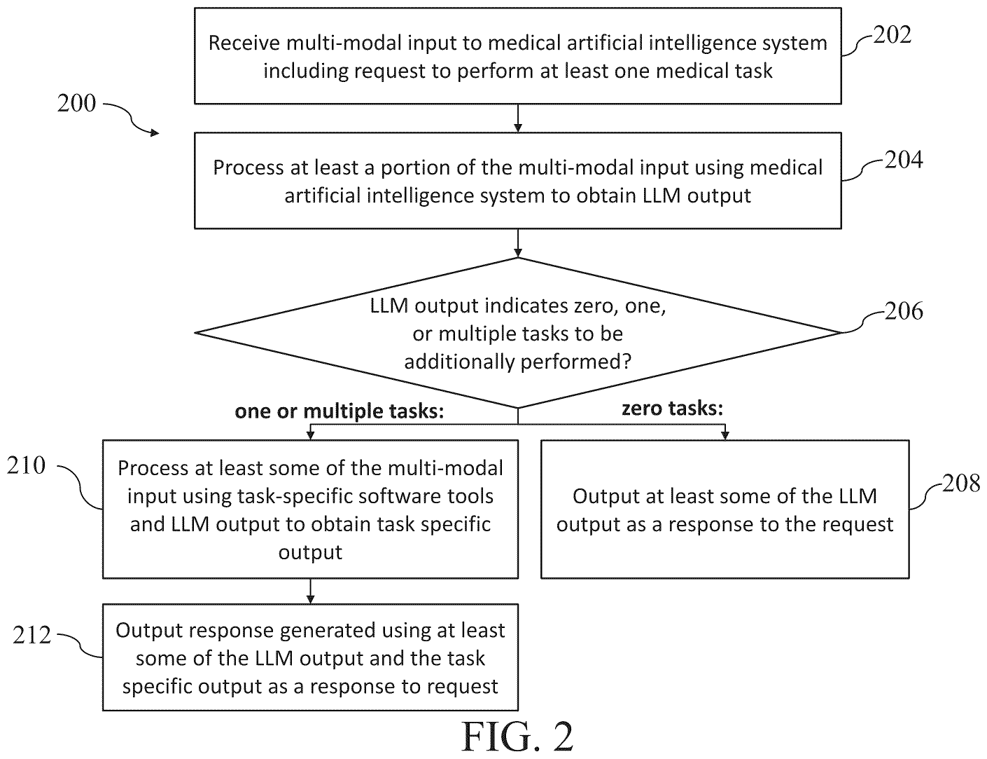

FIG. 2 illustrates an example method for performing medical tasks using the MAI system of FIG. 1, in accordance with some embodiments of the technology described herein. As described herein, the MAI system comprises a trained LLM and a plurality of task specific software tools. The method 200 may be performed using at least one computer hardware processor.

The example method 200 of FIG. 2 begins with act 202 where multi-modal input to an MAI system is received. The multi-modal input includes a request to perform at least one medical task. As described herein, the medical task may be one of a number of tasks, including vision-language tasks and/or vision-centric tasks.

The input to the MAI system at act 202 is multi-modal, examples of which are described herein. The multi-modal input may comprise a text input and an image input, examples of which are described herein.

At act 204, at least a portion of the multi-modal input received at act 202 is processed using the MAI system to obtain an LLM output. For example, the processing may be performed at least in part using the coordinator module 102 and/or the orchestrator module 104 of the MAI system.

At act 206, the LLM output is evaluated to determine whether the LLM output indicates zero, one or multiple tasks are to be additionally performed. If, at act 206, it is determined that zero tasks are to be additionally performed, the method 200 proceeds to act 208, where at least some of the LLM output it output from the MAI system as a response to the request. In this instance, the LLM of the orchestrator module provides the response to the request to perform at least one medical task without using a tool from the specialist tool repository of the MAI system.

If, at act 206, it is determined that one or multiple are to be additionally performed, the method 200 proceeds to act 210 where at least some of the multi-modal input received at act 202 is processed using one or more task-specific software tools from the specialist tool repository and the LLM output to obtain a task specific output. At act 212, a response generated using at least some of the LLM output and the task specific output is output from the MAI system as a response to the request received at act 202.

FIG. 3 illustrates components of the MAI system of FIG. 1, in accordance with some embodiments of the technology described herein. In particular, FIG. 3 illustrates additional aspects of the coordinator module 102, the orchestrator module 104, and the specialist tool repository 106, and how the components of the MAI system 100 interact with each other. For example, FIG. 3 further illustrates an example processing workflow of the MAI system. Dashed arrows indicate that the associated procedures are contingent upon the decision made by the LLM whether to utilize one or more tools from the specialist tool repository. Red arrows (which include all the dashed arrows apart from the “No” branch from the box reciting “If contains <Task>) represent operations undertaken when employing a tool from the specialist tool repository. FIG. 3 illustrates three kinds of tasks: detection, two-dimensional segmentation, and three-dimensional segmentation. These tasks are coded in the MAI system as <DET>, <2DSEG>, and <3DSEG>, respectively.

FIG. 3 illustrates additional aspects of the coordinator module 102. The MAI system may receive multi-modal input, as described herein. The multi-modal input may be in the form of an image-request pair comprising an image input including one or more images and a text input comprising a request (e.g., an indication of a medical task to be performed on the image input using the MAI system). In some embodiments, the vision input may comprise multiple images which may be multi-modal (different types of medical images such as MRI, x-rays, etc.), multiview, and/or multiperiod.

As described herein, the coordinator module 102 processes the input. The coordinator module 102 comprises general vision encoders, vision-language adapters, and a tokenizer. The vision encoder is provided for encoding the image input. The MAI system may autonomously decide whether to use a 2D or 3D vision encoder to process the image input based on an analysis of the input image modality. The 2D vision encoder utilizes a transformer architecture to extract visual tokens from the images. The 3D vision encoder utilizers the architecture of the 3D Unet.

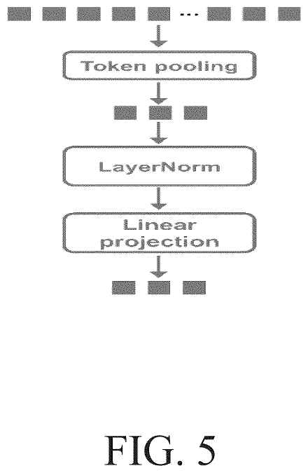

The extracted visual tokens are concatenated and passed to the vision-language adapter for mapping to language space. FIG. 5 illustrates an example architecture of a vision-language adapter of the MAI system of FIG. 1, in accordance with some embodiments of the technology described herein. As shown in FIG. 5, the vision-language adapter includes a stack of three layers. The first layer is responsible for reducing the number of visual tokens to control the GPU memory cost, which can be achieved with an adaptive pooling function. Next, the layer normalization is applied to the pooled visual tokens, followed by a linear projection layer to map the visual representations to the language space. To align with the 2D and 3D vision encoders, two independent adapters are employed to process the extracted visual tokens accordingly.

Meanwhile, the text input comprising request is processed with a tokenizer. The tokenizer may comprise a Llama tokenizer, in some embodiments, which is a byte-pair encoding model based on sentencepiece. The request is transformed into a series of textual tokens, which are then contextualized by the following large language model along with the mapped visual tokens. This enables the system to understand and correlate the visual data with the relevant requests.

Accordingly, the coordinator module 102 allows for processing the multi-modal input to obtain a tokenized representation of the image input and the text input at least in part by processing the text input to generate text tokens and processing the image input to generate visual tokens, and wherein the at least a portion of the multi-modal output comprises the tokenized representation of the image input and the text input. The processing of the multi-modal input to obtain the tokenized representation of the image input and the text input may further comprise adapting the visual tokens into text tokens using a model trained to transform visual tokens into text tokens.

As shown in FIG. 3, the tokenized representation of the image input and the text input is passed to the large language model of the orchestrator module 104. The LLM utilizes the processed output of the coordinator module 102 to generate an LLM output and determine whether to utilize one or more tools of the specialist tool repository 106 to perform one or more additional tasks.

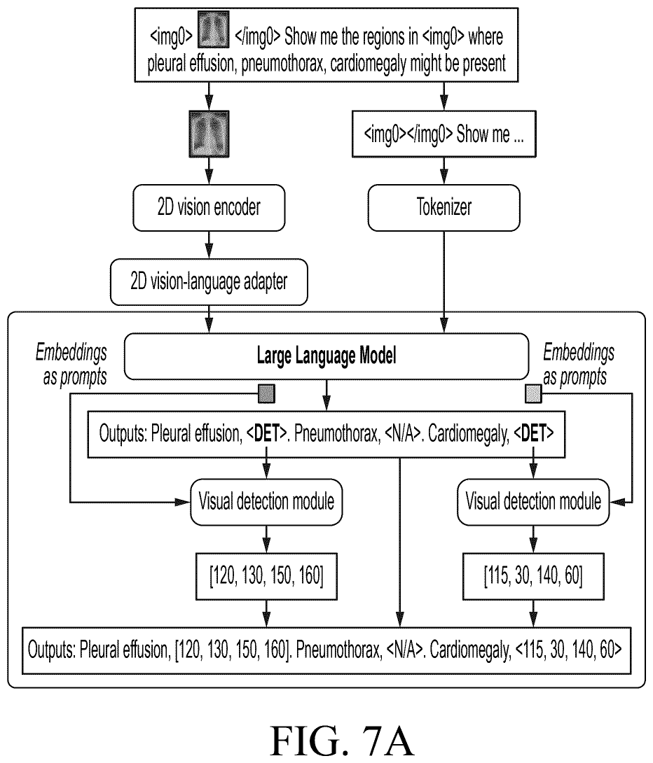

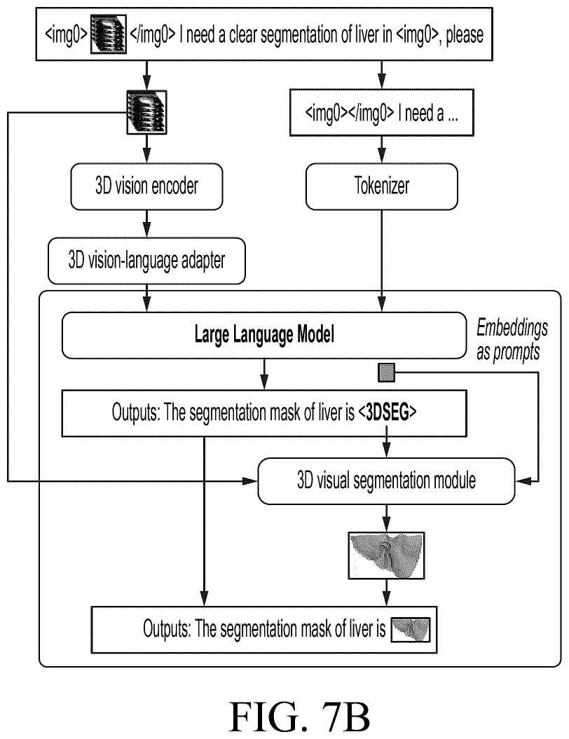

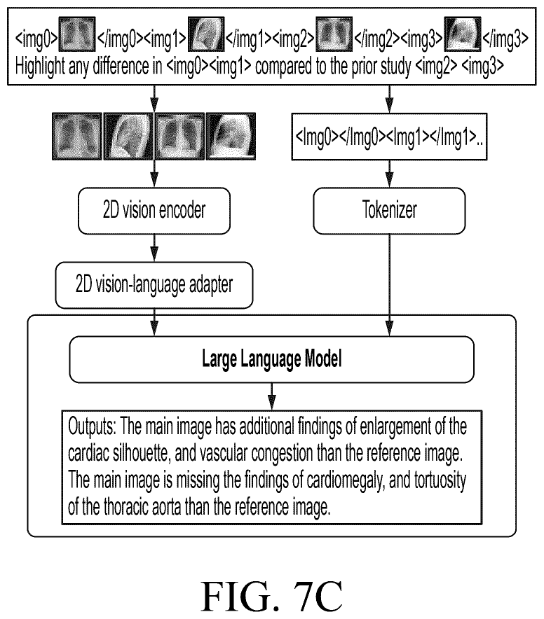

Specifically, the orchestrator module 104 has to decide whether to carry out the task independently or use a specific visual modeling tool from the specialist tool repository 106 for support based on the analysis of visual and linguistic data. This decision-making process can be formulated as: llm_{θ}(I,T)→(llmₒ,sₒk). I and T denote the extracted visual and textual tokens, respectively. llm_{θ} stands for the large language model. llmₒ and sₒk represent the outputs of the language model and the kth specialist tool, respectively. For vision-language tasks, the MAI system only adopts the llmₒ as the final language response. For vision-centric tasks, the choice of k is determined based on the llmₒ. Specifically, the llm_{θ} determines the task type and generates the relevant <Task> in the llmₒ. There are three kinds of <Task> included in MAI system illustrated in FIG. 3: <DET>, <2DSEG>, and <3DSEG>. The predicted <Task> guides the system in selecting the kth visual modeling tool from a specialist tool repository, tailored for executing the task described by <Task> (see FIG. 3 for more details). Meanwhile, the corresponding latent embeddings of <Task> are indexed from the output logits of the lime. These embeddings gather the information from the input data and help prompt the visual modeling tool to complete the desired task. To accomplish this, the indexed latent embeddings can be either passed directly to the visual detection tool or integrated with the intermediate features of the visual segmentation tools. The orchestration process is illustrated on three tasks in FIGS. 7A-C, including the chest pathology detection (FIG. 7A), the abdominal organ segmentation (FIG. 7B), and the longitudinal study comparisons (FIG. 7C).

As shown in FIG. 3, then, the output of the coordinator module 102, that is the tokenized representations of the image and text inputs, are passed to the orchestrator module 104. Latent embeddings are derived from the tokenized representations of the image and text inputs and tokenized, then used to generate a language output. The language output, and specifically the latent embeddings comprising task specifications (<Task>) are evaluated to determine whether to invoke at least one of the task-specific software tools in the specialist tool repository to complete the desired medical task. That is, the LLM determines, based on the latent embedding, whether one or more additional tasks are to be performed. For example, the LLM determines whether the LLM output comprises at least one tag (e.g., a <Task>) associated with a task to be performed by a task-specific software tool. A task-specific software tool to use may be identified by the tag. If it is determined that no additional tasks are to be performed, the orchestrator module outputs the language output as the output to the request.

Alternatively, if it is determined that one or more additional tasks are to be performed, a first task-specific software tool is selected from the specialist tool repository storing the plurality of task-specific software tools. For example, latent embeddings of task specification tokens corresponding to tasks to be additionally performed by the MAI system are indexed and the indexed latent embeddings are encoded into a repository comprising the plurality of task-specific software tools. The indexed embeddings are input to the specialist tool repository 106 as a prompt, along with the image input. A task-specific software tool is selected based on the prompt and the image input is processed using the selected task-specific software tool to generate a task-specific output. The task-specific output may then be integrated output to generate an integrated, or multi-modal, response to the request.

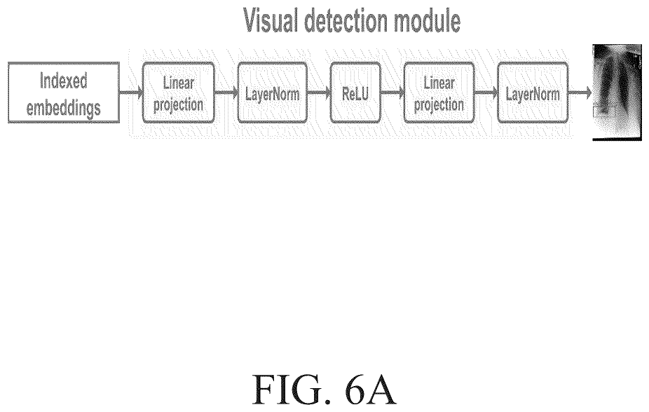

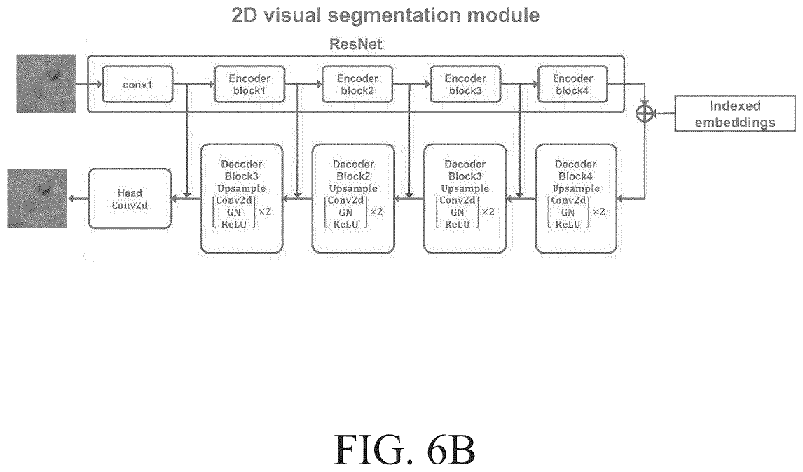

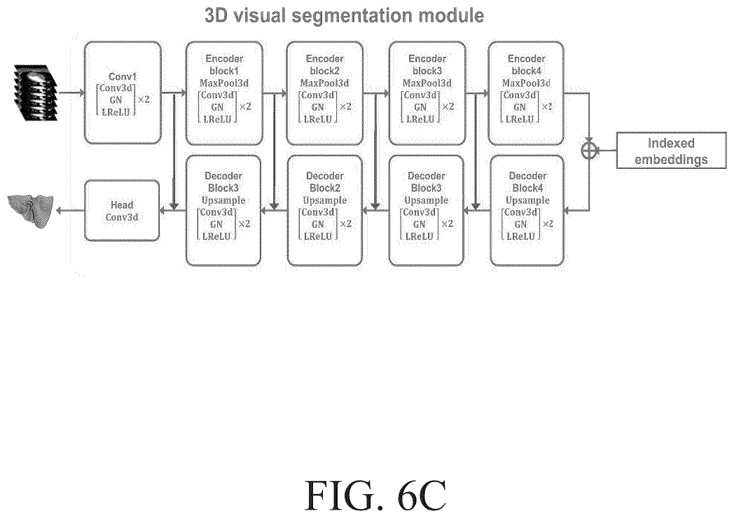

FIGS. 6A-B illustrate examples of tools in the specialist tool repository. For example, FIG. 6A illustrates an example of a visual detection tool, in accordance with some embodiments of the technology described herein. FIG. 6B illustrates an example of a two-dimensional visual segmentation tool, in accordance with some embodiments of the technology described herein. FIG. 6C illustrates an example of a three-dimensional visual segmentation tool, in accordance with some embodiments of the technology described herein. In the figures, Conv2d and Conv3d stand for the 2D and 3D convolution, respectively; GN denotes the group normalization layer, and LReLU represents the leaky ReLU activation function. The encoder of the specialist tool for 2D segmentation may be initialized, for example, using the pretrained weights of ResNet-18 on ImageNet. The red arrows (extending downwards vertically) denote the skip connections.

It should be appreciated that the MAI system, and in particular, the specialist tool repository 106 may be updated to adapt the MAI system to perform different medical tasks. For example, the specialist tool repository may be updated by adjusting how one or more of the plurality of task-specific software tools performs a task and/or adding one or more additional task-specific software tools to the repository. In this way, the MAI system can be easily adapting as the need for performance of different types of medical tasks evolves.

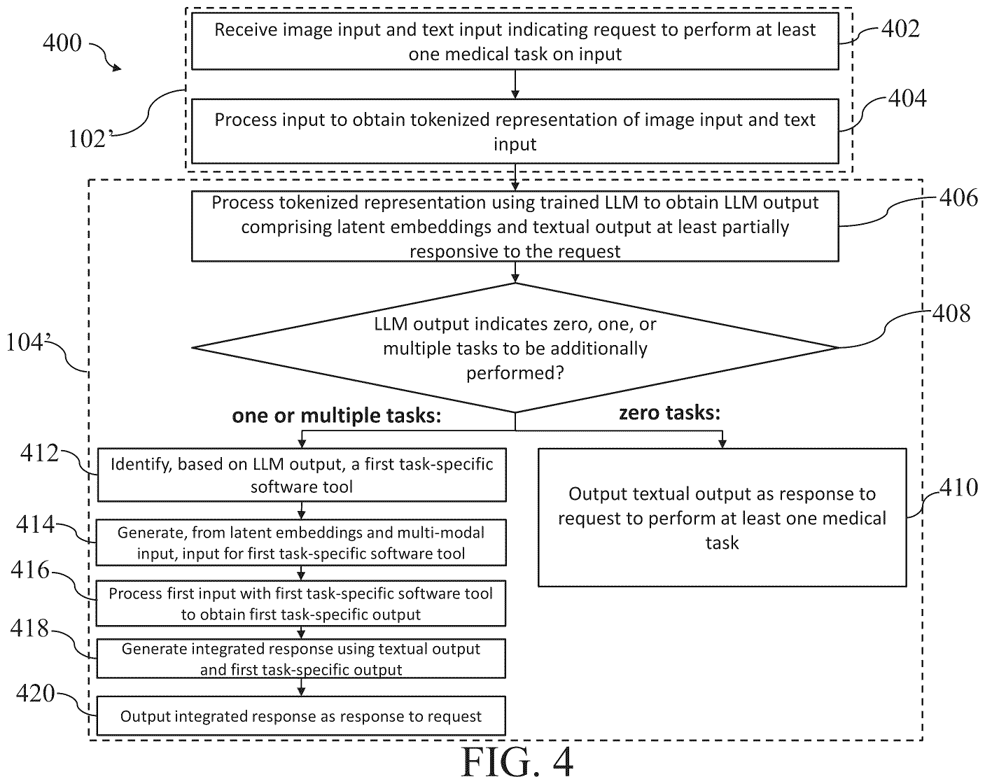

FIG. 4 illustrates another example method for performing medical tasks using the MAI system of FIG. 1, in accordance with some embodiments of the technology described herein. For example, the method 400 illustrated in FIG. 4 expands on the method 200 illustrated in FIG. 2. In FIG. 4, dashed box 102′ illustrates acts that may be performed with the coordinator module 104 and dashed box 104′ illustrates acts that may be performed with the orchestrator module 104, including, in some embodiments, one or more tools from the specialist tool repository 106.

As described herein, the method 400 may be performed by the MAI system 100 described herein. Accordingly, the MAI system performing the method 400 may comprise a plurality of modules including a multi-modal input coordinator module, an orchestrator module comprising a trained LLM, and a plurality of task-specific software tools. The plurality of task-specific software tools (including the first task-specific software tool described herein) may comprise one or more task-specific trained machine learning models. The method 400 may be performed by executing the modules using at least one computer hardware processor to perform the acts 402-418 of the method 400.

At act 402, multi-modal input is received. The multi-modal input may comprise image input and text input. The text input may indicate a request that the MAI system perform at least one medical task on the multi-modal input.

At act 404, the multi-modal input is processed to obtain tokenized representations of the image input and the text input. As described herein, acts 402-404 may be performed using the coordinator module 102 described herein. Accordingly, acts 402 may be performed using the capabilities of the coordinator module 102 described herein (e.g., using the 2D/3D vision encoder, the 2D/3D vision-language adapter, and/or the tokenizer).

In some embodiments, processing the multi-modal input to obtain the tokenized representation of the image input and the text input comprises processing the text input to generate text tokens and processing the image input to generate visual tokens. In some embodiments, processing the multi-modal input to obtain the tokenized representation of the image input and the text input further comprises adapting the visual tokens into text tokens using a model trained to transform visual tokens into text tokens. In some embodiments, when the image input comprises a two-dimensional (2D) image, the processing the image input comprises processing the 2D image using a 2D vision encoder. In some embodiments, when the image input comprises a three-dimensional (3D) image, the processing the image input comprises processing the 3D image using a 3D vision encoder.

At act 406, the tokenized representation obtained at act 404 is processed using a trained LLM (e.g., the trained LLM of the orchestrator module). An LLM output at least partially responsive to the request received at act 402 is obtained by the processing at act 404. The LLM output comprises latent embeddings and textual output and the LLM output indicates zero, one, or multiple tasks are to be additionally performed by at least one of the plurality of task-specific software tools.

At act 408, it is determined whether the LLM output obtained at act 408 indicates zero, one, or multiple tasks are to be additionally performed by the at least one of the plurality of task-specific software tools. In some embodiments, determining whether the LLM output indicates zero, one, or multiple tasks are to be additionally performed by the at least one of the plurality of task-specific software tools comprises determining whether the textual output comprises at least one tag associated with at least one respective task.

If it is determined, at act 408, that zero tasks are to be additionally performed by the at least one of the plurality of task-specific software tools, the method 400 proceeds to act 410 where the textual output of the LLM output is output from the MAI system as a response to the request of the MAI system. If, on the other hand, it is determined that the LLM output indicates that one or multiple tasks are to be additionally performed by the at least one of the plurality of task-specific software tools, the method 400 proceeds to act 412 where a first task-specific software tool is identified, based on the LLM output and from among the plurality of task-specific software tools, a first task-specific software tool. For example, in some embodiments, identifying the first task-specific software tool comprises identifying the first task-specific software tool using the at least one tag, wherein the at least one tag comprises a first tag associated with a first task, and the first task-specific software tool is trained to perform the first task.

At act 414, a first input for the first task-specific software tool is generated from the latent embeddings and the multi-modal input (e.g., the image input). At act 416, the first input is processed with the first task-specific software tool to obtain a first task-specific output.

At act 418, an integrated response is generated using the textual output produced by the trained LLM and the first task-specific output obtained at act 416. At act 420, the integrated output generated at act 418 is output from the MAI system is output as a response to the request of the MAI system.

As described herein, the image input may comprise one or more medical images comprising one or more radiographs, dermoscopy images, and/or computed tomography scans. In some embodiments, the image input comprises one or more two-dimensional medical images, one or more three-dimensional images, and/or one or more videos. In some embodiments, the image input comprises a single image. In some embodiments, the image input comprises multiple images. In some embodiments, the multiple images comprise multiple views of at least a portion of patient. In some embodiments, the multiple images comprise a same view of at least a portion of a patient at multiple points in time.

As described herein, the integrated output may comprise a multi-modal output. In some embodiments, the multi-modal output comprises a text output and an image output. In some embodiments, the textual output of the LLM output comprises the text output and the first task-specific output comprises the image output. In some embodiments, the response to the request of the MAI system comprises one or more of a medical report, a segmented image, an indication of a classified medical condition, a comparison of longitudinal study images, an indication of a detected anatomical structure and/or an indication of a detected abnormality.

As described herein, the at least one medical task to be performed on the input may be one of a variety of medical tasks. For example, in some embodiments, the at least one medical task comprises one or more vision-language tasks comprising one or more of medical report generation, longitudinal study comparison, region-of-interest captioning, open-ended visual question answering, and/or abnormality classification and/or one or more vision-centric tasks comprising one or more medical image analysis tasks including one or more of anatomical structure identification, abnormality characterization, chest abnormality detection, skin lesion segmentation, and/or organ segmentation.

FIG. 7A illustrates an example workflow for performing a chest pathology detection task using the MAI system of FIG. 1, in accordance with some embodiments of the technology described herein. FIG. 7A shows an illustrative example of the workflow shown in FIG. 3 where the task to be performed is a chest pathology detection task.

As shown in the example of FIG. 7A, the image input comprises a two-dimensional image. The LLM output indicates that a detection task is to be additionally performed. For example, the tag <DET> is provided. The at least one of the plurality of task-specific software tools comprises a task-specific software tool trained to perform the detection task on the two-dimensional image. The at least one task-specific output comprises a second textual output. Obtaining the at least one task-specific output comprises generating the second textual output. The response from the MAI system comprises the LLM output and the second textual output, wherein outputting the response comprises outputting the LLM output and the second textual output.

FIG. 7B illustrates an example workflow for performing an abdominal organ segmentation task using the MAI system of FIG. 1, in accordance with some embodiments of the technology described herein. FIG. 7B shows an illustrative example of the workflow shown in FIG. 3 where the task to be performed is an abdominal organ segmentation task.

As shown in the example of FIG. 7B, the image input comprises a three-dimensional image. The LLM output indicates that a segmentation task is to be additionally performed by the at least one of the plurality of task-specific software tools. For example, the tag <3DSEG> is provided. The at least one of the plurality of task-specific software tools comprises a task-specific software tool trained to perform the segmentation task on the three-dimensional image. The at least one task-specific output comprises an image output. Obtaining the at least one task-specific output comprises generating the image output. The response from the MAI system comprises the image output and the LLM output, wherein outputting the response comprises outputting the LLM output and the image output.

FIG. 7C illustrates an example workflow for performing a longitudinal study comparisons task using the MAI system of FIG. 1, in accordance with some embodiments of the technology described herein. FIG. 7C shows an illustrative example of the workflow shown in FIG. 3 where the task to be performed is a longitudinal study.

As shown in FIG. 7C, the image input comprises a plurality of two-dimensional images. The LLM output indicates that zero additional tasks are to be additionally performed by the at least one of the plurality of task-specific software tools. Accordingly, the output of the MAI system in response to the request is the LLM output comprising textual output.

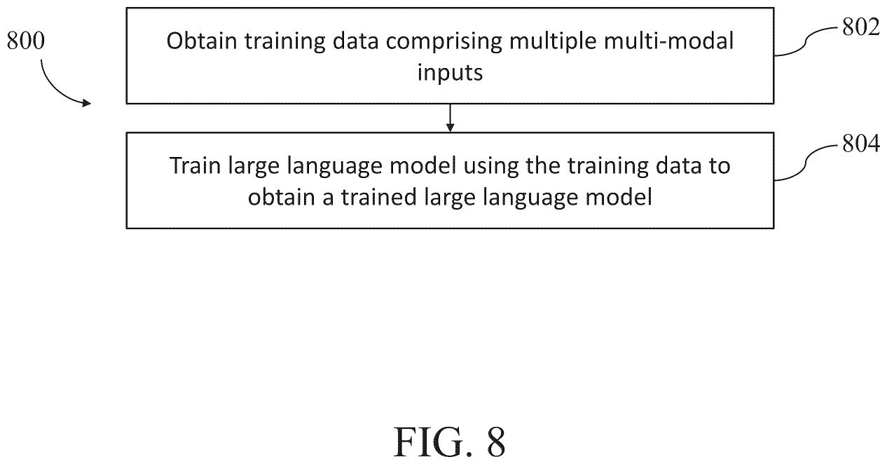

Some aspects of the technology described herein provide for training an MAI system to perform medical tasks. For example, FIG. 8 illustrates an example method for training an MAI system to perform medical tasks, in accordance with some embodiments of the technology described herein. The example method 800 may be performed on MAI system 100 described herein. For example, the MAI system on which method 800 is performed may be comprise an LLM and a plurality of task-specific software tools, wherein the LLM is to be trained to process multi-modal input, containing image input and a request that the MAI system perform at least one medical task on the multi-modal input, to obtain corresponding LLM output at least partially responsive to the request and indicating that zero, one, or multiple tasks are to be additionally performed by at least one of the plurality of task-specific software tools.

The example method 800 begins at act 802 where training data comprising multiple multi-modal inputs is obtained. Each particular one of the multiple multi-modal inputs may comprise a respective image input and a respective request that the MAI system perform at least one respective medical task on the particular multi-modal input.

Training the LLM may be performed using a curated dataset of training data. The training data may comprise a data set including radiographs, dermoscopy images, computed tomography scans, and the medical text data. The training data may span multiple tasks. The training data may include medical images from multiple modalities (e.g., radiographs, dermoscopy images, and computed tomography images). The training data may include clinical language data. In some embodiments, the training data may be processed prior to training the LLM with the training data.

At act 804, the LLM is trained using the training data to obtain a trained LLM. The trained LLM may subsequently be used, for example, to perform at least one medical task as described herein.

Training the LLM may be performed using a plurality of tags, each tag of the plurality of tags is associated a respective task of the tasks to be additionally performed by the at least one of the plurality of task-specific software tools. In some embodiments, the training may comprise optimizing the LLM using domain-aware minibatch gradient descent. In some embodiments, the optimizing the LLM using the domain-aware minibatch gradient descent creates homogeneous minibatches for training, each of the homogenous minibatches specific to a single type of task or tasks to be performed by the orchestrator or at least one of the plurality of task-specific software tools and a single type of imaging modality.

In some embodiments, training the LLM to obtain a trained LLM may be performed using referring image instructions, which enclose the visual tokens of different images with different text identifiers and incorporating the text identifiers into text instructions to help the LLM specify different images. Referring image instruction tuning involves adding image identifiers to instructions to specify different images. This technique enhances the model's capability to perform complex comparative analyses, such as the longitudinal study comparisons, where it is needed to assign images to different studies and compare studies instead of images.

The example processes described herein may be performed using at least one computer hardware processor. For example, aspects of the technology described herein include at least one non-transitory computer-readable storage medium storing instructions that, when executed by at least one computer hardware processor, cause the at least one computer hardware processor to perform any of the processes described herein for performing a medical task using an MAI system and/or for training an MAI system to perform medical tasks. Aspects of the technology described herein further include a system comprising the at least one computer hardware processor and the at least one non-transitory computer-readable storage medium described herein.

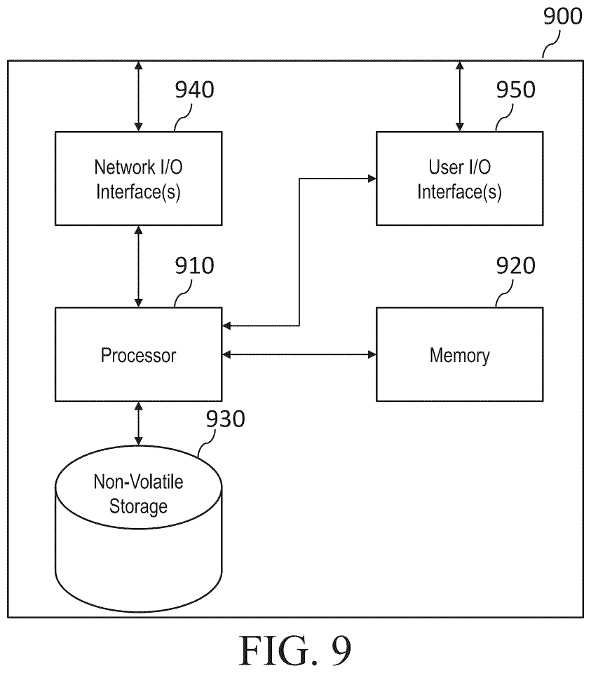

An illustrative implementation of a computer system 900 that may be used in connection with any of the embodiments of the technology described herein (e.g., such as the process of FIG. 1, for example) is shown in FIG. 9. The computer system 900 includes one or more processors 910 and one or more articles of manufacture that comprise non-transitory computer-readable storage media (e.g., memory 920 and one or more non-volatile storage media 930). The processor 910 may control writing data to and reading data from the memory 920 and the non-volatile storage device 930 in any suitable manner, as the aspects of the technology described herein are not limited to any particular techniques for writing or reading data. To perform any of the functionality described herein, the processor 910 may execute one or more processor-executable instructions stored in one or more non-transitory computer-readable storage media (e.g., the memory 920), which may serve as non-transitory computer-readable storage media storing processor-executable instructions for execution by the processor 910.

Computing device 900 may also include a network input/output (I/O) interface 940 via which the computing device may communicate with other computing devices (e.g., over a network), and may also include one or more user I/O interfaces 950, via which the computing device may provide output to and receive input from a user. The user I/O interfaces may include devices such as a keyboard, a mouse, a microphone, a display device (e.g., a monitor or touch screen), speakers, a camera, and/or various other types of I/O devices.

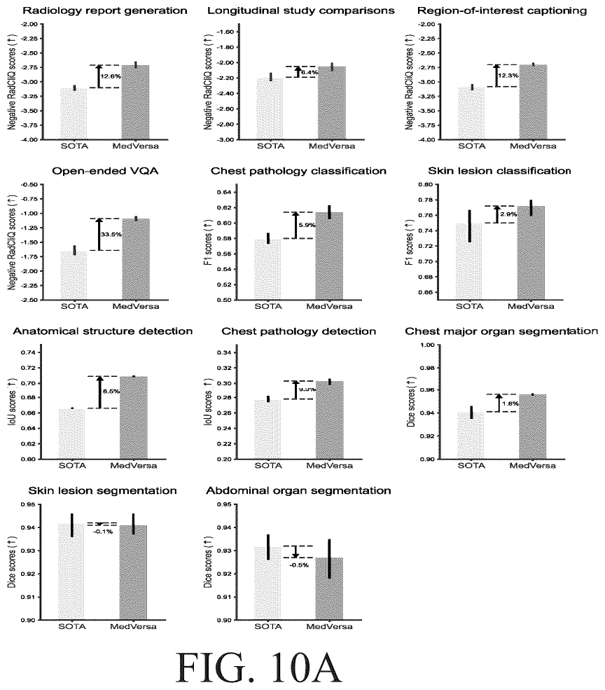

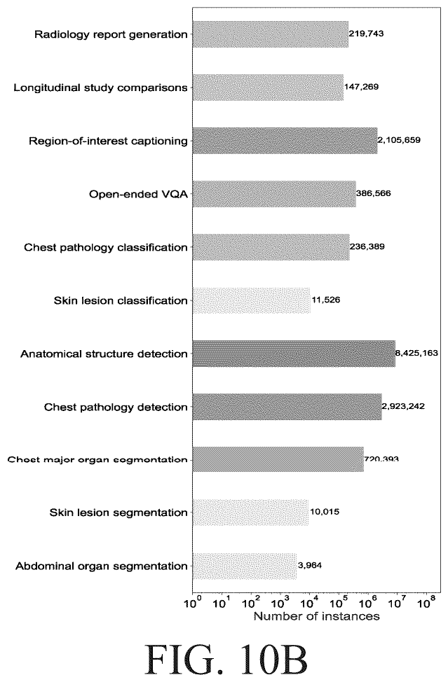

The inventors have recognized that the MAI system described herein outperforms existing systems, such as task-specific systems, on performance of various medical tasks. FIGS. 10A-12B illustrate experimental results of the MAI system's performance on various medical tasks. Further description of the performance of the MAI system is provided in the examples herein.

FIGS. 10A-B illustrate relative improvement of the MAI system described herein over eleven different medical tasks. FIG. 10A illustrates example data showing improvement of the MAI system described herein over existing models across eleven tasks, in accordance with some embodiments of the technology described herein. FIG. 10A shows that the MAI system achieves an over 5% improvement in performing seven of the eleven medical tasks. FIG. 10B illustrates aspects of the data used to train the MAI system described herein, in accordance with some embodiments of the technology described herein. In particular, FIG. 10B illustrates the number of instances of each medical task included in the training data used to train the MAI model described herein. The graph shown in FIG. 10B presents the data distributions across different task settings.

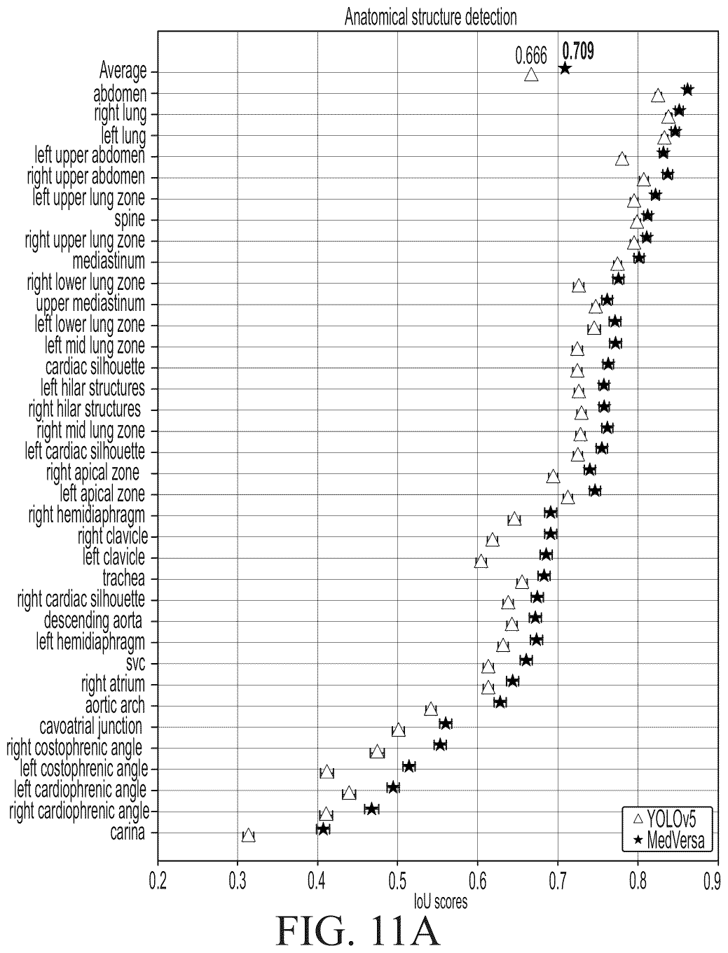

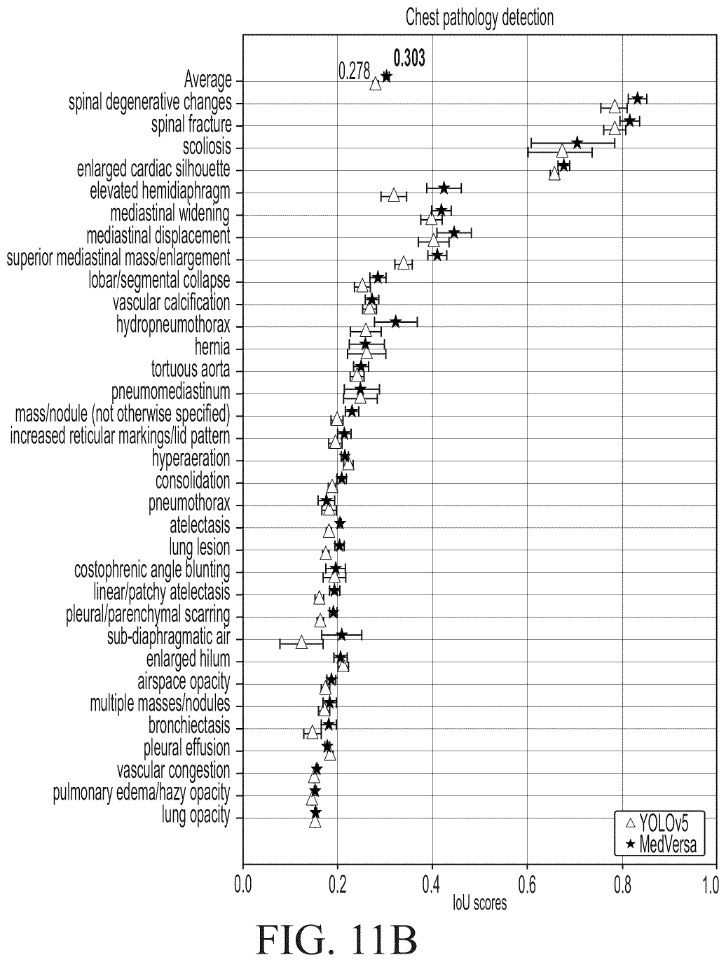

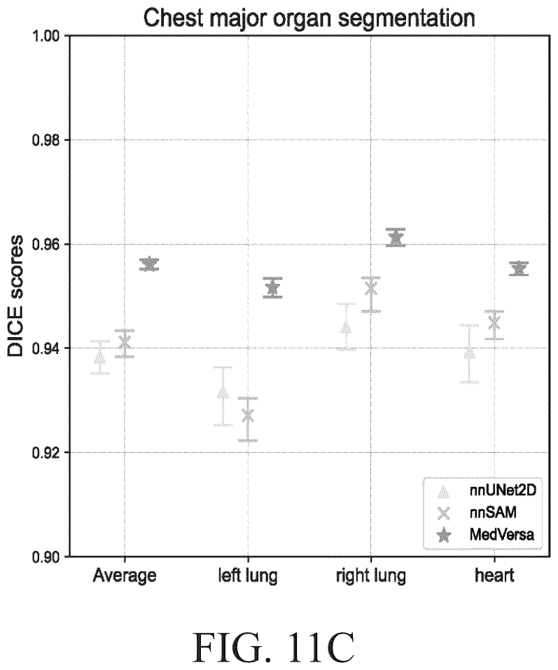

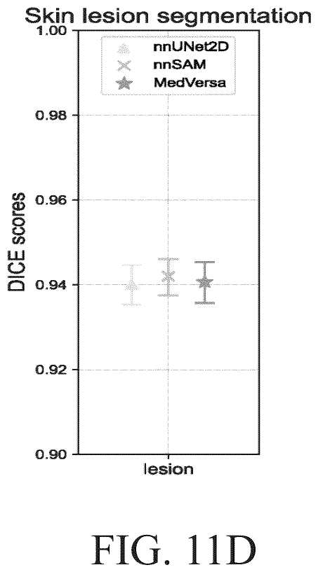

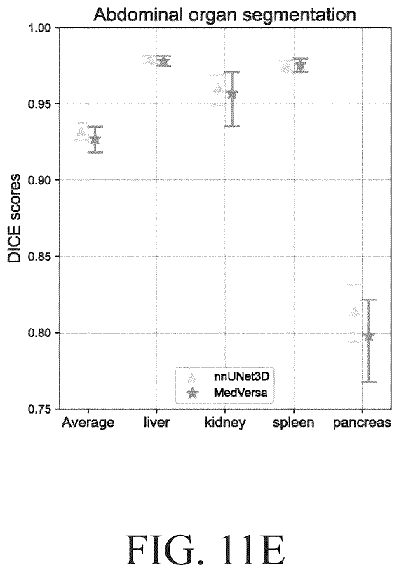

FIGS. 11A-E illustrate experimental results of performance of the MAI system described herein on different vision-centric tasks. FIG. 11A illustrates example results of performance of the MAI system described herein on an anatomical structure detection task, in accordance with some embodiments of the technology described herein. FIG. 11B illustrates example results of performance of the MAI system described herein on a chest pathology detection task, in accordance with some embodiments of the technology described herein. FIG. 11C illustrates example results of performance of the MAI system described herein on a chest major organ segmentation task, in accordance with some embodiments of the technology described herein. FIG. 11D illustrates example results of performance of the MAI system described herein on a skin lesion segmentation task, in accordance with some embodiments of the technology described herein. FIG. 11E illustrates example results of performance of the MAI system described herein on an abdominal organ segmentation task, in accordance with some embodiments of the technology described herein.

The performance of the MAI system described herein is compared to an existing system (YOLOv5) for two detection tasks shown in FIGS. 11A-B and to an existing system (nnSAM) for two-dimensional segmentation tasks shown in FIGS. 11C-D. For medical image segmentation, the MAI system performance was largely compared to nnUNet (nnUNet2D for chest major organ segmentation and skin lesion segmentation shown in FIGS. 11C-D and nnUNet3D for abdominal organ segmentation shown in FIG. 11E). The evaluation metrics of detection and segmentation tasks are Intersection over Union (IoU) and DICE similarity scores. Each of FIGS. 11A-E present a 95% confidence interval.

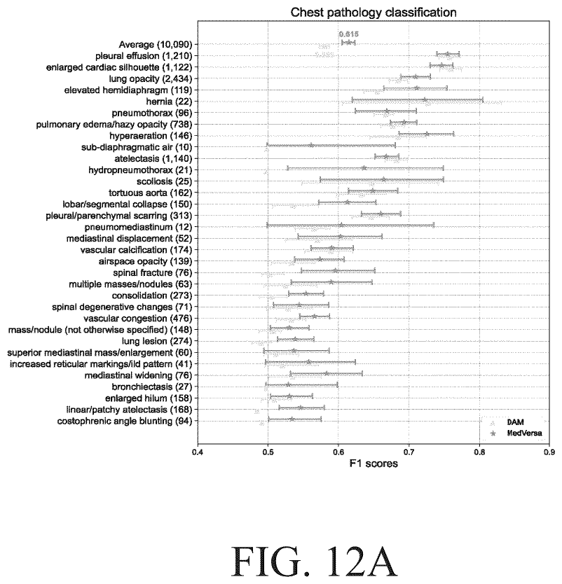

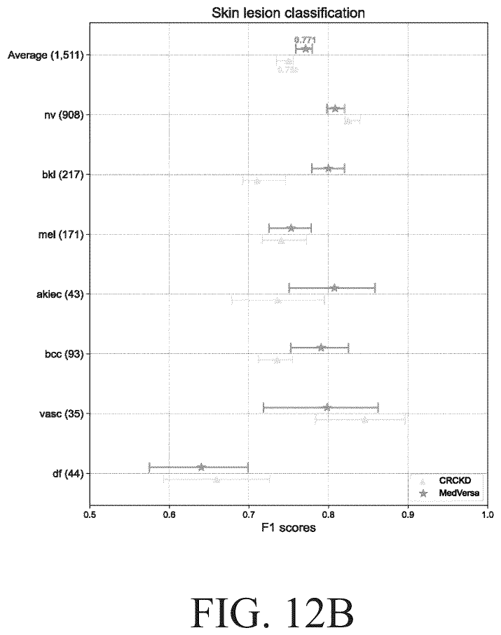

FIGS. 12A-B illustrate experimental results of performance of the MAI system on classification tasks. FIG. 12A illustrates example results of performance of the MAI system described herein on a chest pathology classification task, in accordance with some embodiments of the technology described herein. FIG. 12B illustrates example results of performance of the MAI system described herein on a skin lesion classification task, in accordance with some embodiments of the technology described herein. For the results shown in FIGS. 12A-B illustrating performance of the MAI system for a chest pathology classification task (FIG. 12A) and a skin lesion classification task (FIG. 12B), the MAI system performance was compared to different specialist models including DAM (Deep AUC Maximization) and CRCKD (Categorical Relation-preserving Contrastive Knowledge Distillation). A 95% confidence interval is provided along with F1 scores.

The above-described embodiments can be implemented in any of numerous ways. For example, the embodiments may be implemented using hardware, software, or a combination thereof. When implemented in software, the software code can be executed on any suitable processor (e.g., a microprocessor) or collection of processors, whether provided in a single computing device or distributed among multiple computing devices. It should be appreciated that any component or collection of components that perform the functions described above can be generically considered as one or more controllers that control the above-discussed functions. The one or more controllers can be implemented in numerous ways, such as with dedicated hardware, or with general purpose hardware (e.g., one or more processors) that is programmed using microcode or software to perform the functions recited above.

In this respect, it should be appreciated that one implementation of the embodiments described herein comprises at least one computer-readable storage medium (e.g., RAM, ROM, EEPROM, flash memory or other memory technology, CD-ROM, digital versatile disks (DVD) or other optical disk storage, magnetic cassettes, magnetic tape, magnetic disk storage or other magnetic storage devices, or other tangible, non-transitory computer-readable storage medium) encoded with a computer program (i.e., a plurality of executable instructions) that, when executed on one or more processors, performs the above-discussed functions of one or more embodiments. The computer-readable medium may be transportable such that the program stored thereon can be loaded onto any computing device to implement aspects of the techniques discussed herein. In addition, it should be appreciated that the reference to a computer program which, when executed, performs any of the above-discussed functions, is not limited to an application program running on a host computer. Rather, the terms computer program and software are used herein in a generic sense to reference any type of computer code (e.g., application software, firmware, microcode, or any other form of computer instruction) that can be employed to program one or more processors to implement aspects of the techniques discussed herein.

The foregoing description of implementations provides illustration and description but is not intended to be exhaustive or to limit the implementations to the precise form disclosed. Modifications and variations are possible in light of the above teachings or may be acquired from practice of the implementations. In other implementations the methods depicted in these figures may include fewer operations, different operations, differently ordered operations, and/or additional operations. Further, non-dependent blocks may be performed in parallel.

It will be apparent that example aspects, as described above, may be implemented in many different forms of software, firmware, and hardware in the implementations illustrated in the figures. Further, certain portions of the implementations may be implemented as a “module” that performs one or more functions. This module may include hardware, such as a processor, an application-specific integrated circuit (ASIC), or a field-programmable gate array (FPGA), or a combination of hardware and software.

Having thus described several aspects and embodiments of the technology set forth in the disclosure, it is to be appreciated that various alterations, modifications, and improvements will readily occur to those skilled in the art. Such alterations, modifications, and improvements are intended to be within the spirit and scope of the technology described herein. For example, those of ordinary skill in the art will readily envision a variety of other means and/or structures for performing the function and/or obtaining the results and/or one or more of the advantages described herein, and each of such variations and/or modifications is deemed to be within the scope of the embodiments described herein. Those skilled in the art will recognize or be able to ascertain using no more than routine experimentation many equivalents to the specific embodiments described herein. It is, therefore, to be understood that the foregoing embodiments are presented by way of example only and that, within the scope of the appended claims and equivalents thereto, inventive embodiments may be practiced otherwise than as specifically described. In addition, any combination of two or more features, systems, articles, materials, kits, and/or methods described herein, if such features, systems, articles, materials, kits, and/or methods are not mutually inconsistent, is included within the scope of the present disclosure.

The above-described embodiments can be implemented in any of numerous ways. One or more aspects and embodiments of the present disclosure involving the performance of processes or methods may utilize program instructions executable by a device (e.g., a computer, a processor, or other device) to perform, or control performance of, the processes or methods. In this respect, various inventive concepts may be embodied as a computer readable storage medium (or multiple computer readable storage media) (e.g., a computer memory, one or more floppy discs, compact discs, optical discs, magnetic tapes, flash memories, circuit configurations in Field Programmable Gate Arrays or other semiconductor devices, or other tangible computer storage medium) encoded with one or more programs that, when executed on one or more computers or other processors, perform methods that implement one or more of the various embodiments described above. The computer readable medium or media can be transportable, such that the program or programs stored thereon can be loaded onto one or more different computers or other processors to implement various ones of the aspects described above. In some embodiments, computer readable media may be non-transitory media.

The terms “program” or “software” are used herein in a generic sense to refer to any type of computer code or set of computer-executable instructions that can be employed to program a computer or other processor to implement various aspects as described above. Additionally, it should be appreciated that according to one aspect, one or more computer programs that when executed perform methods of the present disclosure need not reside on a single computer or processor, but may be distributed in a modular fashion among a number of different computers or processors to implement various aspects of the present disclosure.

Computer-executable instructions may be in many forms, such as program modules, executed by one or more computers or other devices. Generally, program modules include routines, programs, objects, components, data structures, etc. that perform particular tasks or implement particular abstract data types. Typically the functionality of the program modules may be combined or distributed as desired in various embodiments.

Also, data structures may be stored in computer-readable media in any suitable form. For simplicity of illustration, data structures may be shown to have fields that are related through location in the data structure. Such relationships may likewise be achieved by assigning storage for the fields with locations in a computer-readable medium that convey relationship between the fields. However, any suitable mechanism may be used to establish a relationship between information in fields of a data structure, including through the use of pointers, tags or other mechanisms that establish relationship between data elements.

When implemented in software, the software code can be executed on any suitable processor or collection of processors, whether provided in a single computer or distributed among multiple computers.

Further, it should be appreciated that a computer may be embodied in any of a number of forms, such as a rack-mounted computer, a desktop computer, a laptop computer, or a tablet computer, as non-limiting examples. Additionally, a computer may be embedded in a device not generally regarded as a computer but with suitable processing capabilities, including a Personal Digital Assistant (PDA), a smartphone, a tablet, or any other suitable portable or fixed electronic device.

Also, a computer may have one or more input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. As another example, a computer may receive input information through speech recognition or in other audible formats.

Such computers may be interconnected by one or more networks in any suitable form, including a local area network or a wide area network, such as an enterprise network, and intelligent network (IN) or the Internet. Such networks may be based on any suitable technology and may operate according to any suitable protocol and may include wireless networks, wired networks or fiber optic networks.

Also, as described, some aspects may be embodied as one or more methods. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

Various aspects of the present invention may be used alone, in combination, or in a variety of arrangements not specifically discussed in the embodiments described in the foregoing and is therefore not limited in its application to the details and arrangement of components set forth in the foregoing description or illustrated in the drawings. For example, aspects described in one embodiment may be combined in any manner with aspects described in other embodiments.

Various events/acts are described herein as occurring or being performed at a specified time. One of ordinary skill in the art would understand that such events/acts may occur or be performed at approximately the specified time.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

The terms “substantially”, “approximately”, and “about” may be used to mean within ±20% of a target value in some embodiments, within ±10% of a target value in some embodiments, within ±5% of a target value in some embodiments, within ±2% of a target value in some embodiments. The terms “approximately” and “about” may include the target value.

Having thus described several aspects of at least one embodiment of this invention, it is to be appreciated that various alterations, modifications, and improvements will readily occur to those skilled in the art.

Such alterations, modifications, and improvements are intended to be part of this disclosure, and are intended to be within the spirit and scope of the invention. Further, though advantages of the present invention are indicated, it should be appreciated that not every embodiment of the invention will include every described advantage. Some embodiments may not implement any features described as advantageous herein and in some instances. Accordingly, the description herein and drawings are by way of example only.

### EXAMPLES

### Example 1: A Generalist Learner for Multifaceted Medical Image Interpretation

Current medical artificial intelligence systems are often limited to narrow applications, hindering their widespread adoption in clinical practice. To address this limitation, MedVersa, a generalist learner that enables flexible learning and tasking for medical image interpretation, is proposed. By leveraging a large language model as a learnable orchestrator, MedVersa can learn from both visual and linguistic supervision, support multimodal inputs, and perform real-time task specification. This versatility allows MedVersa to adapt to various clinical scenarios and perform multifaceted medical image analysis. MedInterp, the largest multimodal dataset to date for medical image interpretation, consisting of over 13 million annotated instances spanning 11 tasks across 3 modalities, is introduced to support the development of MedVersa. The experiments conducted demonstrate that MedVersa achieves state-of-the-art performance in 9 tasks, sometimes outperforming specialist counterparts by over 10%. MedVersa is the first to showcase the viability of multimodal generative medical AI in implementing multimodal outputs, inputs, and dynamic task specification, highlighting its potential as a multifunctional system for comprehensive medical image analysis. This generalist approach to medical image interpretation paves the way for more adaptable and efficient AI-assisted clinical decision-making.

### INTRODUCTION

The field of medical artificial intelligence (AI) has been advancing at a rapid pace, ushering in a new era of diagnostic accuracy and patient care. Within this dynamic landscape, researchers have been focusing their efforts on developing solutions for specific tasks, such as identifying chest pathologies (Rajpurkar et al. 2017; Wang et al. 2017; Irvin et al. 2019; Johnson et al. 2019; Tiu et al. 2022) and classifying skin diseases. (Liu et al. 2020; Esteva et al. 2017; Daneshjou et al. 2022) Similarly, the majority of medical AI products approved by the US Food and Drug Administration for clinical use have been designed to address one or two specific tasks. (Joshi et al. 2022) However, this task-specific approach may limit the real-world clinical applications of these AI systems, as they may not be able to adapt to the diverse and complex needs of healthcare settings. (Moor et al. 2023; Rajpurkar et al. 2023)

Addressing this concern, generalist medical artificial intelligence (GMAI) was proposed to utilize recent advanced in foundation models (Bommasani et al. 2021) for more flexible problem solving. (Moor et al. 2023) However, contemporary GMAI models have been designed to learn from natural language supervision. (Tu et al. 2023; Wu et al. 2023; Moor et al. 2023; Lu et al. 2023; Huang et al. 2023) Although these models work well in vision-language tasks, it does not readily apply to a majority of vision-centric problems, such as detection and segmentation, which are indispensable to medical image interpretation. (Chen et al. 2022; Wang et al. 2023; Zhang et al. 2022)

Inspired by the GMAI paradigm, MedVersa, a generalist learner capable of multifaceted medical image interpretation is proposed. At the core of MedVersa is to function the large language model as a learnable orchestrator, which learns to orchestrate the multimodal inputs and execute tasks using language/vision modules. This architectural design equips MedVersa to overcome the limitations of traditional approaches (Tu et al. 2023; Wu et al. 2023; Moor et al. 2023; Lu et al. 2023; Huang et al. 2023) by integrating both visual and linguistic supervision within its learning processes, while at the same time supporting on-the-fly task specification with language. MedVersa is a versatile model that excels in both vision-language tasks, such as generating radiology reports and answering visual questions, and vision-centric challenges, including detecting anatomical structures and segmenting medical images (FIG. 1). This dual capability enables MedVersa to train on diverse medical data across multiple modalities and tasks, resulting in general, shared representations.

To facilitate the development of MedVersa, a diverse and multimodal dataset called MedInterp, which is specifically designed for multifaceted medical image interpretation, was designed (FIGS. 10A-B). MedInterp is an extensive dataset, containing over 13 million annotated instances and covering a wide range of vision-language and vision-centric tasks. By training and assessing MedVersa on the MedInterp dataset, it was demonstrated that MedVersa surpasses state-of-the-art specialist counterparts in nine tasks, often by notable margins (FIGS. 10A-B). For instance, in the task of radiology report generation, MedVersa outperforms both MAIRA-1 (Hyland et al. 2023) a specialist large multimodal model from Microsoft, and Med-PaLM M (Tu et al. 2023), a generalist biomedical foundation model from Google that is 10 times larger than MedVersa. Moreover, MedVersa also excels in visual localization tasks, surpassing the well-established object detector (Jocher et al. 2020) in two localization tasks. Additionally, MedVersa demonstrates superior performance compared to state-of-the-art specialist models in various other tasks, including longitudinal study comparisons, region-of-interest captioning, open-ended VQA, and chest pathology classification. The model's consistent and superior performance has been validated on six external cohorts, highlighting its robustness and generalizability.

### Results

### Report Generation

Table 2 presents the evaluation results on three sections: findings, impression, and target (concatenation of findings and impression) using five evaluation metrics: BLEU-4 (Papineni et al. 2002), BertScore (Zhang et al. 2019), CheXbert (Smit et al. 2020), RadGraph Jain et al. 2021), and RadCliQ (Yu et al. 2023). For the findings section, among the baselines, MAIRA-1 (Hyland et al. 2023) achieves a higher BLEU-4 score of 14.2, while Med-PaLM M Tu et al. 2023) produces a better RadGraph score of 26.7, both of which are the current state-of-the-art. Since MAIRA-1 and Med-PaLM are not publicly accessible, another competitive baseline, ClsGen (Nguyen et al. 2021), was added for consistent comparisons across different sections. ClsGen achieves a higher BLEU-4 score than Med-PaLM M.

TABLE 2
External validation results. Seven capabilities (i.e., report generation, classification,
detection, segmentation, open-ended visual question answering, and region-of-interest
captioning) were evaluated on six unseen external cohorts (i.e., IUX-ray, CheXpert,
NIH ChestX-ray, and MS-CXR). For each capability, MedVersa was compared against
a state-of-the-art specialist model. For classification, detection, and segmentation
tasks, the mean F1 score, mean IoU (Intersection over Union), and mean DICE score
were used as the evaluation metrics, respectively. For other tasks, the results
of RadCliQ were reported. Numbers in brackets are the 95% confidence intervals.
↓ indicates that the lower results are better.

| Capabilities | Datasets | Models | Metrics | Results | 95% CIs |
|---|---|---|---|---|---|
| Report | IUX-ray | ClsGen | RadCliQ (↓) | 3.07 | [3.00, 3.12] |
| generation | | MedVersa | | 2.57 | [2.54, 2.60] |
| Classification | CheXpert | DAM | Mean F1 | 0.653 | [0.633, 0.669] |
| | | MedVersa | score | 0.734 | [0.712, 0.756] |
| Detection | NIH | YOLOv5 | Mean IoU | 0.223 | [0.210, 0.235] |
| | ChestX-ray | MedVersa | | 0.239 | [0.225, 0.254] |
| Segmentation | CheXmask | nnSAM | Mean DICE | 0.923 | [0.917, 0.928] |
| | | MedVersa | score | 0.955 | [0.952, 0.957] |
| Open-ended | IUX-ray | PTLM | RadCliQ (↓) | 1.75 | [1.69, 1.82] |
| VQA | | MedVersa | | 1.12 | [1.07, 1.17] |
| Region-of- | MS-CXR | MiniGPT-v2 | RadCliQ (↓) | 3.43 | [3.38, 3.48] |
| interest | | MedVersa | | 3.29 | [3.23, 3.35] |
| captioning | | | | | |

The proposed MedVersa was evaluated across all sections and metrics. It outperforms all baselines in the findings section with a BLEU-4 score of 17.8 (vs. 14.2 of MAIRA-1), a CheXBert score of 46.4 (vs. 44.0 of MAIRA-1), and a RadGraph score of 28.0 (vs. 26.7 of Med-PaLM M), establishing its superiority and setting the new state-of-the-art in capturing both the linguistic and clinical aspects of radiology reporting. Particularly noteworthy is that the results of Med-PaLM M were obtained from a significantly larger model, with ten times more parameters than those of MedVersa. This implies that the latter model is more advantageous in terms of training and inference efficiency. For the impression section, MedVersa surpasses ClsGen in all evaluation metrics, and the same superiority is maintained when all sections are combined. External validation was performed on the IUX-ray dataset (Demner-Fushman et al. 2016) (Table 2), where MedVersa keeps maintaining a notable advantage over ClsGen.

### Vision-Centric Tasks

For detection tasks, MedVersa exhibits competitive performance, surpassing YOLOv5 (Jocher et al. 2020) by noticeable, consistent margins in the detection of a variety of anatomical structures (FIG. 11A), with most IoU scores on certain structures surpassing 0.6. It shows particularly high effectiveness in the detection of lung zones. When identifying chest pathologies, MedVersa's capabilities exceed those of YOLOv5, notably in the detection of 27 out of 33 conditions (FIG. 11B). It also maintains a higher average performance compared to YOLOv5 (0.303 vs. 0.278). On the external cohort NIH ChestXray, MedVersa also outperforms YOLOv5 by an average of nearly two percent in detecting common chest pathologies (Table 2).

Regarding segmentation tasks, MedVersa demonstrates competitive results, performing competitively to nnUNet (Isensee et al. 2021) and nnSAM (Li et al. 2023). All three approaches perform fairly well in segmenting major chest organs (FIG. 11C) and skin lesions (FIG. 11D).

Nonetheless, MedVersa outperforms nnUNet and nnSAM by significant margins in chest major organ segmentation. In the task of abdominal organ segmentation (FIG. 11E), MedVersa also shows competitive performance to nnUNet3D which uses complex and time-consuming data augmentation techniques. Table 3 showcases the segmentation results of skin lesions and abdominal organs.

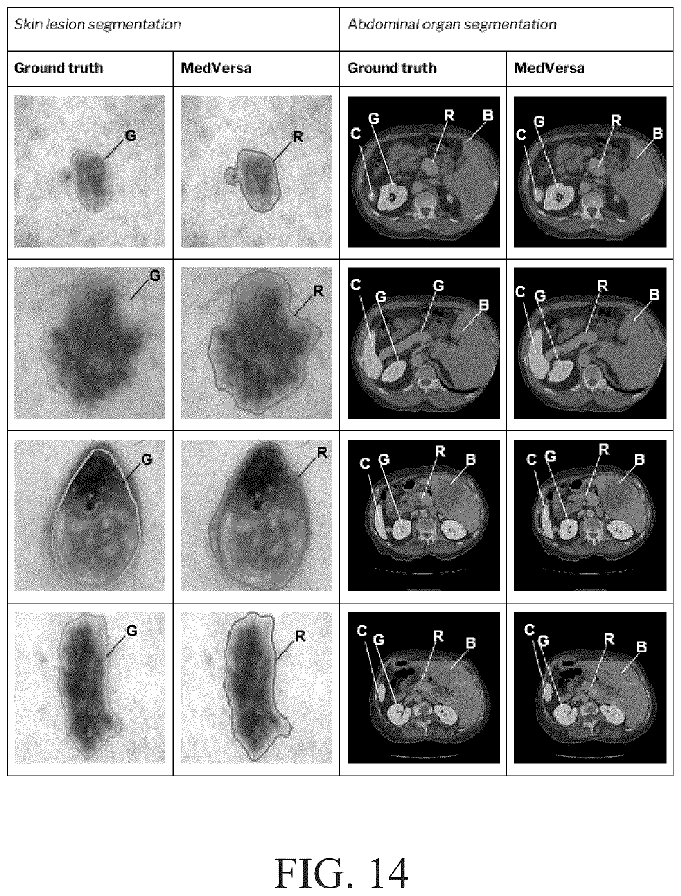

FIG. 14 illustrates Table 3 which shows segmentation results of skin lesions and abdominal organs. For abdominal organ segmentation, the red (R), blue (B), green (G), and aqua (A) colors represent the pancreas, liver, kidney, and spleen, respectively.

### Longitudinal Study Comparisons, Open-Ended VQA, and Region-of-Interest Captioning

In longitudinal study comparisons, the model is typically tasked with drawing a comparative conclusion between two groups of images collected at different periods. This presents a significant challenge for image interpretation, as models must work with multiple images to analyze various anatomical structures, extracting features and identifying subtle disease-related changes. As shown in Table 1, the baseline method EKAID builds complex anatomical structure-aware graphs to encode anatomical and disease features for recognizing the differences between CXR studies. In contrast, MedVersa adopts a straightforward yet effective way to process longitudinal images (see. FIG. 7C). Moreover, MedVersa largely outperforms EKAID across different metrics (BLEU-4: 44.7 vs. 40.4, BertScore: 71.4 vs. 69.1, CheXbert: 50.0 vs. 49.1, RadGraph: 23.7 vs. 20.4, RadCliQ: 2.05 vs. 2.19).

TABLE 1
Experimental results of 4 vision-language tasks: radiology report generation, longitudinal
study comparisons, open-ended visual question answering, and region-of-interest captioning.

| | | Eval. | | | | | |
|---|---|---|---|---|---|---|---|
| Tasks | Models | section | BLEU-4 | BertScore | CheXbert | RadGraph | RadCliQ (↓) |
| Radiology | ClsGen | Findings | 11.9 | 40.5 | 42.6 | 23.5 | 3.28 |
| report | | | [11.4, 12.31 | [39.8, 41.1] | [41.9, 43.4] | [22.8, 24.2] | [3.24, 3.33] |
| generation | MAIRA-1 | Findings | 14.2 | — | 44.0 | 24.3 | 3.10 |
| | | | [13.7, 14.7] | | [43.1, 44.9] | [23.7, 24.8] | [3.07, 3.14] |
| | Med-PaLM | Findings | 11.5 | — | — | 26.7 | — |
| | M (85B) | | [—, —] | | | [—, —] | |
| | MedVersa | Findings | 17.8 | 49.7 | 46.4 | 28.0 | 2.71 |
| | | | [17.2, 18.4] | [49.0, 50.4] | [45.5, 47.4] | [27.3, 28.7] | [2.66. 2.75] |
| | ClsGen | Impression | 8.5 | 38.0 | 48.7 | 18.8 | 3.25 |
| | | | [7.6, 9.3] | [37.3, 38.6] | [48.0, 49.5] | [18.0, 19.7] | [3.18, 3.33] |
| | MedVersa | Impression | 13.7 | 48.9 | 52.4 | 25.7 | 2.66 |
| | | | [12.7, 14.7] | [48.0, 49.8] | [51.3, 53.5] | [24.6, 26.9] | [2.60, 2.71] |
| | ClsGen | Target | 13.7 | 42.4 | 44.3 | 25.2 | 3.20 |
| | | | [13.0, 14.3] | [41.6, 43.1] | [43.2, 45.4] | [24.4, 26.0] | [3.14, 3.25] |
| | MedVersa | Target | 16.0 | 47.4 | 46.6 | 30.0 | 2.74 |
| | | | [15.3, 16.7] | [46.6, 48.2] | [45.3, 47.8] | [29.1, 30.8] | [2.69, 2.79] |
| Longitudinal | EKAID | All | 40.4 | 69.1 | 49.1 | 20.4 | 2.19 |
| study | | | [39.9, 41.0] | [68.7, 69.5] | [48.7, 49.4] | [19.9, 20.9] | [2.14, 2.23] |
| comparisons | MedVersa | All | 44.7 | 71.4 | 50.0 | 23.7 | 2.05 |
| | | | [43.7, 45.6] | [70.6, 72.2] | [49.5, 50.6] | [22.6, 24.9] | [2.01, 2.10] |
| Open- | PTLM | All | 25.2 | 64.7 | 78.3 | 30.4 | 1.64 |
| ended | | | [24.4, 26.0] | [64.1, 65.5] | [77.3, 79.2] | [29.7, 31.0] | [1.57, 1.71] |
| VQA | MedVersa | All | 31.2 | 76.5 | 85.1 | 33.4 | 1.09 |
| | | | [30.7, 31.8] | [75.9, 77.1] | [84.6, 85.6] | [32.7, 34.2] | [1.06, 1.12] |
| Region-of- | MiniGPT- | All | 5.1 | 36.6 | 55.3 | 18.3 | 3.08 |
| interest | v2 | | [4.6, 5.5] | [36.3, 37.0] | [54.9, 55.8] | [17.9, 18.6] | [3.05, 3.13] |
| captioning | MedVersa | All | 8.4 | 43.8 | 60.7 | 22.8 | 2.70 |
| | | | [8.2, 8.7] | [43.6, 44.1] | [60.4, 61.1] | [22.5, 23.1] | [2.68, 2.71] |

Specifically, the evaluation of radiology reports was conducted on three different sections: findings, impression, and target (concatenation).
Results of MAIRA-1 and Med-PaLM M are cited from their papers as their models have not been released.
Numbers in brackets are the 95% confidence intervals.
↓ indicates that the lower results are better.
VQA stands for visual question answering.

As Table 1 displays, MedVersa outperforms PTLM (van Sonsbeek et al. 2023), a state-of-the-art model for open-ended medical VQA, by an average of six percent in BLEU-4, BertScore, CheXbert, and RadGraph scores. MedVersa also achieves a 30% lower RadCliQ score compared to PTLM. The 95% confidence intervals indicate that the improvement brought by MedVersa can be statistically significant. The result on the external cohort also validates the advantage of MedVersa (Table 2).

In the task of region-of-interest captioning, MedVersa shows an obvious advantage over MiniGPT-v2 across various metrics. The RadCliQ score, which comprehensively evaluates the lexical and clinical significance of generated text, is substantially lower for MedVersa at 2.70 versus 3.08 for MiniGPT-v2, suggesting captions of MedVersa are semantically more aligned with reference standards. The result from an external cohort further confirms the benefit of MedVersa, as shown in Table 2.

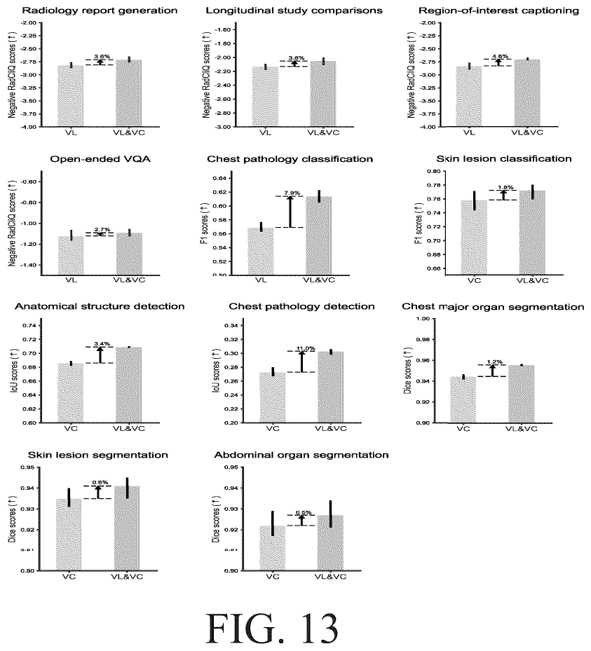

FIG. 13 illustrates comparative analyses of learning from multimodal supervision. The impact of training MedVersa with different types of tasks (i.e., with different forms of supervision) was studied). VC denotes training with vision-centric tasks, while VL denotes training with vision-language data.

### Classification Tasks in Radiology and Dermatology

FIGS. 12A-B present performance comparisons of MedVersa against DAM (Yuan et al. 2021) in chest pathology classification and against CRCKD (Xing et al. 2021) in skin lesion classification, both of which are top performing models in their respective fields. MedVersa demonstrates superior performance over DAM with an average F1 score of 0.615, notably higher than DAM's 0.580 in chest pathology classification (FIG. 12A). This pattern of outperformance extends in 29 out 33 pathologies, including both common (e.g., lung opacity, pulmonary edema, spinal fracture) and less common ones (e.g., hydropneumothorax, bronchiectasis), which indicates MedVersa's strong diagnostic accuracy across various conditions. In skin lesion classification, MedVersa's advantage is also noticeable. The average F1 score of MedVersa is 0.772, appreciably above CRCKD's 0.750, underscoring MedVersa's effectiveness in classifying skin conditions (FIG. 12B). It is worth noting that MedVersa outperforms CRCKD by significant margins in benign keratosis-like lesions (bkl), which has a diverse range of subtypes. This further demonstrates the generalization ability of MedVersa. For external validation (Table 2), MedVersa again surpasses DAM by a large margin on CheXpert (Irvin et al. 2019), which also exceeds the mean performance of radiologists (F1 score: 0.734 vs. 0.610) (Tiu et al. 2022).

### Discussion

The inventors recognize MedVersa to be the first GMAI model that supports multi-modal inputs, outputs, and on-the-fly task specification. Trained on MedInterp, a medical dataset encompassing 11 different tasks across three imaging modalities, MedVersa sets the new state-of-the-art in report generation and outperforms highly competitive specialist models in both vision-language and vision-centric tasks. The development of orchestrated systems unlocks new opportunities to build more versatile GMAI models. More detailed perspectives are provided in the following.

MedVersa integrates visual and linguistic supervision through its multimodal-output design. MedVersa distinguishes itself from previous endeavors by seamlessly incorporating both visual and textual guidance in its training process. This unique approach allows MedVersa to tackle a wide range of medical tasks, from generating radiology reports to segmenting medical images. The model's ability to assimilate knowledge from various input types and generate multimodal outputs results in the development of general and robust shared representations, which helps boost the model accuracy on the tasks and alleviate potential biases in the data. The incorporation of multimodal outputs in MedVersa's also aligns with the latest progress in generative AI, where the use of varied and all-encompassing training data has yielded promising results. By gaining insights from both visual and textual cues, MedVersa constructs a more comprehensive grasp of medical information, paving the way for more precise and dependable diagnoses. Its capacity to adapt to impromptu task specifications renders MedVersa a multifaceted and flexible instrument for diverse clinical applications, establishing its place as a useful resource in medical AI for thorough diagnostics.

Large language models act as orchestrators. Unlike previous endeavors that used large language models as standalone language predictors, the large language model in MedVersa transcends its traditional role by acting as an orchestrator capable of interpreting clinical language data and dynamically coordinating with specialist tools for specific, vision-centric tasks. The integration of specialist tools within O-GMAI enhances its capability in areas where language-based models traditionally falter, such as detailed image analysis required in chest abnormality detection and skin lesion segmentation. The comprehensive approach, combining the contextual decision-making of the large language model with the precision of specialist tools, offers a more robust and versatile diagnostic tool. This new orchestration represents a new step beyond the limitations of previous medical foundation models, offering a new perspective of integrating large language models into generative multi-modal medical AI.

Impact of dataset composition. While the majority of the data used to train MedVersa consists of X-ray images, with a smaller proportion of dermatology (derm) and computed tomography (CT) data, this imbalance does not fundamentally impact the validity of our generalist model training approach. The primary focus is on investigating how to effectively learn from visual and linguistic supervision and how to integrate multiple tasks within a single model. The choice to predominantly use X-ray data was driven by its wide availability and the prevalence of associated text reports, which facilitate the exploration of the research questions. Nevertheless, it is acknowledged that the inclusion of a more diverse range of imaging modalities could potentially enhance the model's generalization capabilities. Future work could explore the impact of incorporating a more balanced dataset with a higher proportion of other imaging modalities, such as derm, CT, and magnetic resonance imaging (MRI). Despite this limitation, it is believed that the current study provides valuable insights into the effectiveness of learning from visual and linguistic supervision and the feasibility of multi-task integration in a generalist model.

Orchestrated modeling enables flexible medical image interpretation. By synthesizing the strengths of the large language model and specialist vision tools, O-GMAI adeptly addresses a broad spectrum of medical tasks, ranging from the generation of radiology reports to the precise segmentation of organs. This unified approach, contrasting with prior models predominantly reliant on large language models, facilitates nuanced and targeted analyses tailored to specific medical tasks. The integration of specialist visual modeling tools within the O-GMAI system is particularly noteworthy for enhancing its performance in vision-centric tasks, a domain where language-based models traditionally exhibit limitations. The proficiency of MedVersa is evident in its ability to accurately detect chest abnormalities and segment skin lesions, which are essential in diagnosing a variety of critical conditions. Furthermore, its effectiveness in handling complex tasks like longitudinal study comparisons and region-of-interest captioning underscores its adaptability. MedVersa, through its flexible medical image interpretation, not only broadens the scope of AI applications in healthcare but also signifies a pivotal step towards more personalized and precise medical diagnostics.

MedVersa integrates strong vision-language and vision-centric capabilities. The advancement of MedVersa as a GMAI model is a crucial step in addressing the complex needs of clinical diagnosis and patient care. Its design, which combines linguistic and visual processing capabilities, enables it to handle both vision-language and vision-centric tasks effectively. This versatility is particularly important in medical settings where diverse data types and diagnostic requirements are common. The performance of MedVersa in radiology report generation, surpassing well-established models like MAIRA-1 and Med-PaLM M, highlights its proficiency in integrating and interpreting multi-modal data. Furthermore, its superiority in visual localization tasks, outdoing the YOLOv5 detector, indicates its potential in tackling vision-centric problems. The superior performance of MedVersa in a variety of vision-language and vision-centric tasks underscores its potential as a valuable tool in medical AI, moving towards more comprehensive diagnostics.

Extensible GMAI and beyond. The O-GMAI system features a notable level of extensibility, allowing for the practical integration of new specialist tools into its existing framework. This aspect of MedVersa enables it to adapt and grow in response to evolving medical imaging techniques and diagnostic requirements. Differing from traditional medical AI models, MedVersa integrates the large language model in the way that provides an extensible platform for the addition of new specialist models as advancements in medical technology occur. This feature ensures that the O-GMAI system remains up-to-date and effective in a field characterized by rapid technological changes and emerging diagnostic challenges. For example, adding a specialist tool for advanced neuroimaging can enhance the diagnostic accuracy of MedVersa in neurological conditions. Likewise, as novel medical imaging methods are introduced, the O-GMAI system can be updated with corresponding specialist models, maintaining its relevance in the dynamic landscape of medical diagnostics. This modular design not only prepares it for future advancements but also encourages ongoing improvement and innovation within the system. It highlights the potential of O-GMAI as an extensible and adaptable solution in medical AI, equipped to address the varied and changing requirements of healthcare practitioners and patients in a continuously evolving medical environment.

### Methods

### Datasets and Data Preprocessing

MedInterp was curated to more comprehensively train and evaluate medical FMs for the purpose of medical image interpretation. An overview of MedInterp is presented in Table 3B. Specifically, MedInterp consists of 10 publicly available datasets, some of which are associated with more than one task.

TABLE 3B
Overview of MedInterp. All datasets included in MedInterp can be accessed and downloaded via the provided
URLs. The dataset size was reported after preprocessing. For each dataset, denoted the associated task(s)
and the stage(s) involved are denoted. VQA denotes visual question answering. 1, 2, 3 in the stages
column stand for the training, internal validation, and external validation stages, respectively.

| Datasets | Size | Tasks | Stages | URL |
|---|---|---|---|---|
| MIMIC-CXR | 216,420 studies | Radiology report generation | 1, 2 | https://physionet.org/content/mimic-cxr/2.0.0/ |
| Chest | 235,721 images | Chest pathology | 1, 2 | https://physionet.org/content/chest- |
| ImaGenome | | classification | | imagenome/1.0.0/ |
| | 8,425,163 boxes | Anatomical structure | 1, 2 | |
| | | detection | | |
| | 2,922,665 boxes | Chest pathology detection | 1, 2 | |
| | 2,104,211 | Region-of-interest | 1, 2 | |
| | captions | captioning | | |
| Medical-Diff- | 383,683 QA | Open-ended VQA | 1, 2 | https://github.com/Holipori/ |
| VQA | pairs | | | MIMIC-Diff-VQA |
| | 147,269 | Longitudinal study | 1, 2 | |
| | comparisons | comparisons | | |
| | 2,883 QA pairs | Open-ended VQA | 3 | |
| HAM10000 | 11,526 images | Skin lesion classification | 1, 2 | https://dataverse.harvard.edu/ |
| | 10,015 masks | Skin lesion segmentation | 1, 2 | dataset.xhtml?persistentId=doi:10.7910/ |
| | | | | DVN/DBW86T |
| AbdomenCT- | 3,964 masks | Abdominal organ | 1, 2 | https://github.com/ |
| 1K | | segmentation | | JunMa11/AbdomenCT-1K |
| CheXmask | 719,793 masks | Chest major organ | 1, 2 | https://physionet.org/ |
| | 600 masks | segmentation | 3 | content/chexmask-cxr- |
| | | | | segmentation-data/0.3/ |
| CheXpert | 668 images | Chest pathology | 3 | https://stanfordmlgroup.github.io/ |
| | | classification | | competitions/chexpert/ |
| IUX-ray | 3,323 studies | Radiology report generation | 3 | https://openi.nlm.nih.gov/ |
| NIH ChestX- | 577 boxes | Chest pathology detection | 3 | https://nihcc.app.box.com/v/Chest |
| ray | | | | Xray-NIHCC |
| MS-CXR | 1,448 captions | Region-of-interest | 3 | https://physionet.org/content/ms- |
| | | captioning | | cxr/0.1/ |

MIMIC-CXR. This is a large, publicly accessible dataset comprising 377,110 chest X-rays (CXRs) corresponding to 227,835 radiographic studies performed at the Beth Israel Deaconess Medical Center in Boston, MA (Johnson et al. 2019). The dataset was fully deidentified, and the protected health information was also removed. The official split Johnson et al. 2019) was referred to and studies with ‘train’ and ‘validate’ tags were combined into the training set, while the rest were included in the test set (for internal validation). The free-text radiology report preprocessing followed the steps in CXR-RePair (Endo et al. 2021). Specifically, sections of indication, comparison, findings, and impression were extracted from free-text radiology reports via keywords matching. Then studies with empty findings and impression sections were filtered out. After these steps, 149,711 (2,144) findings sections and 189,411 (2,212) impression sections were obtained. Numbers in parentheses denote the sample size of the test set. Besides, complete radiology reports, i.e., reports that have findings and impression sections were also extracted. This resulted in 122,702 (1,437) complete reports, which were also involved in training and internal validation stages along with sections of findings and impressions. Note that some studies may have more than one CXR, and images of MIMIC-CXR were also used in other tasks.

Chest ImaGenome. This dataset augmented the free-text reports of MIMIC-CXR (Johnson et al. 2019; Johnson et al. 2019) with local annotations derived from both rule-based natural language processing (NLP) and atlas-based bounding box detection (Wu et al. 2021). These annotations are intricately linked through CXR ontologies developed by radiologists, forming anatomy-centered scene graphs. The data split of MIMIC-CXR was followed to avoid training and test sets leakage. The chest pathology classification task included 235,721 CXRs with annotations of 33 pathologies (FIG. 12A). A vast majority of CXRs have bounding box annotations of 36 anatomical structures (see FIG. 12A), leading to 8,425,163 boxes in total. The anatomy-centered graph-structured annotation of Chest ImaGenome was also exploited. For chest pathology detection, connections between pathologies and anatomies were first identified. Next, bounding boxes of anatomies were assigned to associated pathologies that were marked positive. A similar strategy was also adopted for region-of-interest captioning, where connections between sentences from free-text reports and anatomies were extracted using NLP techniques (Wu et al. 2021). After this, textual captions grounded on anatomies were provided. The task input would be the box coordinates of anatomies, and the output would be the associated captions.

Medical-Diff-VQA. This is a publicly available dataset containing a vast number of question-answer pairs based on CXRs (Hu et al. 2023). To construct this dataset, keywords of abnormality and their attributes were first collected. Then, regular expressions were utilized to detect abnormality/disease keywords within the free-text reports of each patient visit in MIMIC-CXR (Johnson et al. 2019; Johnson et al. 2019). These identified keywords served as anchor terms to segment the sentences, and nearby text sections were then scanned for the relevant attribute keywords. The accuracy and completeness of the extracted information have been carefully checked by humans and advanced NLP tools (Hu et al. 2023). In practice, the code in an open source repository was leveraged to generate the datasets (Hu et al. 2023). Since open-ended visual question answering is a main focus, the number of yes/no question-answer pairs were reduced by setting the ‘less_yes_no’ variable in the code to True. This results in 383,683 normal question-answer pairs, where each pair is associated with one frontal CXR, and 147,269 longitudinal comparisons, where each comparison encompasses 2 studies, and each study may contain more than one CXR. The same data split as in Chest ImaGenome and MIMIC-CXR were used to avoid training and test information leakage across different datasets. To build a cohort for external validation, the dataset construction code was applied to the free-text reports of IUX-ray (Demner-Fushman et al. 2016) to extract 2,883 normal question-answer pairs.

HAM10000. This is a bulk collection of multi-source dermatoscopic images of common pigmented skin lesions (Tschandl et al. 2018). The dataset comprises 10,015 image cases and encompasses a diverse collection of significant diagnostic categories within the domain of pigmented lesions. These categories include Actinic keratoses and intraepithelial carcinoma/Bowen's disease (akiec), basal cell carcinoma (bcc), benign keratosis-like lesions (bkl), dermatofibroma (df), melanoma (mel), melanocytic nevi (nv), and vascular lesions (vasc). For the skin lesion classification task, 1,511 images from ISIC 2018 task three were used as the test set for internal validation (Codella et al. 2019). For skin lesion segmentation, the dataset was randomly split into training and test sets. The ratio of the training set to the test set is 9:1. Skin classification models were trained on the raw datasets directly without any class balancing skills (Alam et al. 2022).

AbdomenCT-1K. This is a collection of abdominal CT scans, constructed to enhance existing single-organ datasets with annotations of four abdominal organs annotations: liver, kidney, spleen, and pancreas (Ma et al. 2022). 991 scans with publicly available segmentation masks were used for model training and validation. They were randomly split into training and validation sets, the ratio between which is 9:1. Considering the large amount of GPU memory cost for training 3D segmentation networks, each scan was resized to a 3D volume sized 192 (width)×192 (height)×64 (depth) pixels. The Hounsfiled Unit values were also clipped at [−200, 300].

CheXmask. The is a large-scale dataset of anatomical segmentation masks for multi-center chest radiographs. In practice, CXRs (239,931 images) were incorporated from MIMIC-CXR (Johnson et al. 2019; Johnson et al. 2019) for model training and internal validation, while the validation set (200 images) of CheXpert (Irvin et al. 2019) was used for external validation. Each CXR was associated with segmentation masks of three major chest organs: left lung, right lung, and heart.

CheXpert. This is another large public dataset for chest radiograph interpretation, which retrospectively collected the chest radiographic examinations from Stanford Hospital, performed between October 2002 and July 2017 (Irvin et al. 2019). In this case, its test set (500 studies, 668 images) was used with strong ground truth for externally validating the results of the chest pathology classification task. The test set labels were established through the majority vote of annotations from five radiologists, with three of them being the same as those who annotated the validation set, while the other two were randomly selected. Besides, 200 images in the validation set of CheXpert were also used in the task of chest major organ segmentation.

IUX-ray. The dataset contains 7,470 pairs of CXRs and radiology reports Demner-Fushman et al. 2016). It served as the external validation set for the report generation task. To maintain the consistency of cross-dataset validation, reports that do not contain sections of findings and impression simultaneously were filtered out, resulting in 3,323 studies. Each study has one frontal and one lateral CXRs, associated with one radiology report. Note that images of IUX-ray were also used in the external validation of the open-ended visual question answering task.

NIH ChestX-ray. This dataset includes over 100,000 anonymized CXRs of more than 30,000 individuals from the NIH Clinical Center (Wang et al. 2017). Apart from image-level pathology labels, NIH ChestX-ray also provides a small number of bounding box annotations. In practice, the box annotations (577 boxes) of four common chest pathologies—atelectasis, cardiomegaly, effusion, and pneumothorax—were incorporated into the external validation set for chest pathology detection.

MS-CXR. The dataset offers phrase grounding annotations that are locally aligned by board-certified radiologists, aiming to support research in the domain of complex semantic modeling for biomedical vision-language tasks (Boecking et al. 2022; Boecking et al. 2022). Each phrase is associated with at least one bounding box annotated on one CXR. For the region-of-interest captioning task, MS-CXR was used as the external validation cohort, where box coordinates were passed to the model to generate descriptive text.

### Pipeline

As shown in FIG. 3, MedVersa is composed of three components: the multi-modal input coordinator, the large language model based orchestrator, and the specialist tool repository that contains a variety of visual modeling tools. In practical usage, MedVersa expects inputs in the form of image-request pairs. Note that the vision input may consist of more than one image, which can be multi-modal, multiview or multiperiod (see FIG. 1). MedVersa autonomously decides whether to use a 2D or a 3D vision encoder to process the vision inputs based on an analysis of the input modality. After receiving the processed inputs, the large language model can decide whether to independently perform the task or utilize a set of visual modeling tools from the ‘specialist tool repository’ for assistance. This dynamic decision-making process ensures that tasks are handled with the appropriate level of expertise and efficiency.

### MedVersa, an Orchestrated GMAI System

Multi-modal input coordinator. As FIG. 3 displays, the multi-modal input coordinator comprises the general vision encoders, the vision-language adapters, and the tokenizer. This architecture was designed by taking inspirations from MiniGPT-4 (Zhu et al. 2023; Chen et al. 2023), LLaVA-Med (Li et al. 2023), and Med-PaLM M (Tu et al. 2023) but the architecture was kept easy for implementation. The general vision encoders are the primary gate for vision inputs. Specifically, distinct encoders are exploited for 2D and 3D imaging data, respectively. The 2D vision encoder utilizes the transformer architecture (Vaswani et al. 2017) to extract visual tokens from the images. For the 3D encoder, the encoder from the 3D UNet (Ciçek et al. 2016) is referred to. The extracted visual tokens are concatenated and passed to the adapter to get mapped to the language space. Here, an efficient design of the vision-language adapter is presented, which only contains a stack of three layers (FIG. 5). The first layer is responsible for reducing the number of visual tokens to control the GPU memory cost, which can be achieved with an adaptive pooling function (He et al. 2015). Next, the layer normalization (Ba et al. 2016) is applied to the pooled visual tokens, followed by a linear projection layer to map the visual representations to the language space. To align with the 2D and 3D vision encoders, two independent adapters are employed to process the extracted visual tokens accordingly. Meanwhile, the paired request is processed with the Llama tokenizer (Touvron et al. 2023), which is a byte-pair encoding model based on sentencepiece (Kudo and Richardson 2018). The request is transformed into a series of textual tokens, which are then contextualized by the following large language model along with the mapped visual tokens. This enables the system to understand and correlate the visual data with the relevant requests.

Orchestrated modeling. Unlike prior research that depended exclusively on large language models (LLMs) for task execution, O-GMAI leverages the planning capabilities of the large language model to act as an orchestrator of system operations. Specifically, the orchestrator has to decide whether to carry out the task independently or use a specific visual modeling tool for support based on the analysis of visual and linguistic data. This decision-making process can be formulated as: llm_{θ}(I,T)→(llmₒ,sₒk). I and T denote the extracted visual and textual tokens, respectively. llm_{θ} stands for the large language model. llmₒ and sₒk represent the outputs of the language model and the kth specialist tool, respectively. For vision-language tasks, MedVersa only adopts the llmₒ as the final language response. For vision-centric tasks, the choice of k is determined based on the llmₒ. Specifically, the llm_{θ} determines the task type and generates the relevant <Task> in the llmₒ. There are three kinds of <Task> included in MedVersa: <DET>, <2DSEG>, and <3DSEG>. The predicted <Task> guides the system in selecting the kth visual modeling tool from a specialist tool repository, tailored for executing the task described by <Task> (see FIG. 3 for more details). Meanwhile, the corresponding latent embeddings of <Task> are indexed from the output logits of the llm_{θ}. These embeddings gather the information from the input data and help prompt the visual modeling tool to complete the desired task. To accomplish this, the indexed latent embeddings can be either passed directly to the visual detection tool or integrated with the intermediate features of the visual segmentation tools. The orchestration process is illustrated on three tasks in FIGS. 7A-C, including the chest pathology detection (FIG. 7A), the abdominal organ segmentation (FIG. 7B), and the longitudinal study comparisons (FIG. 7C).

Specialist tool repository. In our tool collection, three specialist models designed are incorporated for vision-focused tasks, and these can be readily expanded or replaced if additional or new specialist tools become necessary. As shown in FIG. 6A, a lightweight visual detection tool was developed that can be integrated with the orchestrator. For the visual segmentation tools, 2D (Isensee et al. 2018) and 3D UNets (Ciçek et al. 2016) are employed for 2D and 3D image segmentation tasks, respectively. The encoder of the 2D UNet is initialized using the pretrained weights of ResNet-18 (He et al. 2015) on ImageNet (Deng et al. 2009). Several different approaches were attempted to incorporate the indexed embeddings from the large language model into the specialist tools. The inventors have observed that the feature concatenation or addition outperforms the more complex operation, such as cross attention (Jaegle et al. 2021). Based on this, the indexed embeddings were added to the intermediate feature maps in segmentation tools, while feeding these embeddings to the detection tool directly. Note that all specialist tools are learnable and need to be trained with the other parts of MedVersa.

Model training and testing with meticulous, referring image instructions. The success of Alpaca, along with recent advancements in large language models (Taori et al. 2023; Wei et al. 2021; Chung et al. 2022; Singhal et al. 2023), have underscored the importance of incorporating diverse instructions to consolidate multiple tasks and enhance generalization capabilities during supervised fine-tuning. This compelling evidence prompted embracing this concept within MedVersa.

Referring image instruction tuning is proposed, where image identifiers are added to instructions to specify different images. This technique enhances the model's capability to perform complex comparative analyses, such as the longitudinal study comparisons, where it is needed to assign images to different studies and compare studies instead of images. For example, in FIG. 7C, the exact input to MedVersa for longitudinal study comparisons is like:
“<img0>v₀</img0><img1>v₁</img1><img2>v₂</img2><img3>v₃</img3> Highlight any difference in <img0><img1> compared to the prior study <img2><img3>.”
vᵢ stands for the visual tokens of the ith input image. All instructions used in the model training are showcased in Table 4. For each task in the study, ChatGPT was used to generate a maximum of 20 prompts, each adhering to a predefined template. This template consists of the initial instruction for each task, providing a structured starting point for the prompts. After this, a manual review was conducted, carefully sifting through the generated prompts to eliminate any that were similar in nature, ensuring that only the most diverse and distinct prompts were retained for the analysis. During the training and test phases, for a given sample corresponding to a specific task, an instruction was chosen at random from the set of instructions linked to the task.

TABLE 4
Instructions used by MedVersa in different tasks. _*_ is the placeholder for the
main image identifier (e.g., <img0>). -*- denotes the placeholder for the abnormalities,
bounding box coordinates, reference image identifier, detection, and segmentation targets
in binary classification, region-of-interest captioning, longitudinal study comparisons,
detection, and segmentation tasks, respectively. For each task, ChatGPT was used to generate
at most 20 prompts based on a predefined template (i.e., the first instruction for each
task). Then, similar prompts were manually filtered out and diverse prompts were retained.

| Tasks | Instructions |
|---|---|
| Report generation | 1. Can you detail the findings observed in _*_? |
| (findings section) | 2. Kindly enumerate the findings from _*_. |
| | 3. I'd like a breakdown of the findings from _*_. |
| | 4. I'd like a section on the findings derived from _*_. |
| | 5. Please write a finding section for _*_. |
| | 6. Would you please write a finding section for _*_? |
| | 7. Please write a section of findings for _*_. |
| | 8. Would you please write a section of findings for _*_? |
| | 9. How would you characterize the findings from _*_? |
| | 10. Please list the discernible findings from _*_? |
| | 11. Can you compile a list of all the notable findings present in _*_? |
| | 12. Please document any findings you see in _*_. |
| Report generation | 1. Can you please provide your overall impression of _*_? |
| (impression section) | 2. What's your main impression from _*_? |
| | 3. Please draft a concise impression on _*_. |
| | 4. Would you give a comprehensive impression based on _*_? |
| | 5. I'm looking for an impression for _*_. |
| | 6. Provide your diagnostic impression based on the _*_. |
| | 7. Draft an impression for _*_. |
| | 8. Would you please write an impression section for _*_? |
| | 9. Summarize the impression for _*_. |
| Report generation | 1. Can you provide a radiology report for _*_? |
| (complete report) | 2. Please report _*_. |
| | 3. Can you provide a report of _*_ with findings and impression? |
| | 4. Report _*_ with findings and impression. |
| | 5. Please write a radiology report for _*_. |
| | 6. Please generate a radiology report for _*_. |
| | 7. Please provide a detailed report for _*_. |
| | 8. Can you provide a comprehensive report of _*_? |
| | 9. Please write a radiology report for _*_. |
| | 10. Can you give a thorough report of _*_? |
| | 11. Could you please report _*_? |
| | 12. Can you provide a comprehensive report for _*_? |
| Chest pathology | 1. What is the diagnosis for _*_? |
| classification, skin | 2. Based on _*_, what type of lung disease is suspected? |
| lesion classification | 3. Can you identify any abnormality in _*_? |
| | 4. What pathology is indicated by _*_? |
| | 5. What lung disease is likely present in _*_? |
| | 6. What are your conclusions from _*_? |
| | 7. What is your interpretation result of _*_? |
| | 8. What abnormalities are present in _*_? |
| | 9. What is the differential diagnosis for the findings in _*_? |
| Region-of-interest | 1. Describe region -*- in _*_. |
| captioning | 2. Detail any abnormalities in -*- of _*_. |
| | 3. Can you characterize the features within -*- on _*_? |
| | 4. Please provide an analysis of the anomalies seen in -*- within _*_. |
| | 5. Describe any pathological findings within -*- of _*_. |
| | 6. Highlight and explain any abnormalities you detect in -*- of _*_. |
| | 7. Identify and describe any abnormality in -*- of _*_. |
| | 8. Could you please describe the region -*- in _*_? |
| | 9. Would you please describe the region -*- in _*_? |
| | 10. Give a description of the region -*- in _*_. |
| Longitudinal study | 1. Highlight any difference in _*_ compared to the prior study -*-. |
| comparisons | 2. Identify any progression in _*_ since the last study -*-. |
| | 3. Compare the current study _*_ with the past one -*- and identify any |
| | difference between them. |
| | 4. Present any changes in _*_ since the last study -*-. |
| | 5. Detail any progression or regression in _*_ in comparison to the older |
| | study -*-. |
| | 6. Detect changes in _*_ compared to the past study -*-. |
| | 7. Compare _*_ with the prior study -*- and tell me any difference. |
| Anatomical | 1. Detect any signs of -*- in _*_. |
| structure detection, | 2. Highlight the areas that indicate -*- in _*_. |
| chest pathology | 3. Show me the regions in _*_ where -*- might be present. |
| detection | 4. Assess _*_ and mark areas consistent with -*- findings. |
| | 5. Locate and circle any features of -*- in _*_. |
| | 6. Compare _*_ to typical -*- patterns and highlight any matches. |
| | 7. Detect and display potential symptoms of -*- within _*_. |
| | 8. Is there any trace of -*- in _*_? Point it out. |
| | 9. Help me spot -*- by illuminating its markers in _*_. |
| | 10. Search for any characteristic signs of -*- in _*_. |
| | 11. Examine and underscore the presence of -*- in _*_. |
| | 12. Would you please help me locate -*- in _*_? |
| | 13. Could you please help me locate -*- in _*_? |
| | 14. Please help me locate -*- in _*_? |
| Chest major organ | 1. Segment -*- in _*_. |
| segmentation, skin | 2. Highlight the boundaries of -*- in _*_. |
| lesion segmentation, | 3. Isolate and show only -*- from _*_. |
| abdominal organ | 4. Can you delineate -*- in _*_? |
| segmentation | 5. Segment -*- from the given _*_. |
| | 6. I need a clear segmentation of -*- in _*_, please. |
| | 7. Outline the contours of -*- in _*_. |
| | 8. Show a clear boundary around -*- in _*_. |
| | 9. Separate -*- from the surrounding anatomy in _*_. |
| | 10. Provide a segmented view of -*- in _*_. |
| | 11. Please identify and segment -*- from the rest in _*_. |
| | 12. Give me a clear cutout of -*- in _*_. |
| | 13. Please mask everything except for -*- in _*_. |
| | 14. Draw a boundary around -*- in _*_. |
| | 15. Would you please help me segment -*- in _*_? |
| | 16. Could you please help me segment -*- in _*_? |
| | 17. Please help me segment -*- in _*_? |

Here, the method for creating ground truth labels is outlined for various tasks and samples. For vision-language tasks, natural language answers are directly utilized as the target for training the model. In particular, for classification tasks, the model is instructed to produce the names of diagnoses. When dealing with vision-centric tasks, distinct labeling techniques are employed for detection and segmentation. In detection tasks, during each training iteration, up to nine classes at random were initially selected and their names (together with a randomly chosen instruction) were conveyed to the model. The model is trained to append either <N/A> or <DET> tags following each class name. <N/A> indicates the absence of the corresponding class in the input image, whereas <DET> signifies its presence. Subsequently, the latent embeddings of <DET> tags are identified and used in the visual detection tool for determining bounding box coordinates. For segmentation tasks, a single class name is randomly chosen from the set, and this name, along with the instruction, is fed into the model. The model is then trained to generate either “The segmentation mask of [class name] is <2DSEG>” or “The segmentation mask of [class name] is <3DSEG>,” depending on whether the task is 2D or 3D segmentation. As in detection, the relevant embeddings for <2DSEG> or <3DSEG> are then passed to the appropriate 2D or 3D visual segmentation tools to create the segmentation masks.

Domain-aware minibatch gradient descent for multi-modal multitask training. Unlike current medical FMs (Tu et al. 2023; Wu et al. 2023; Moor et al. 2023) that only probed the vision-language capability, MedVersa needs to be trained on both vision-language and vision-centric tasks, which brings challenges to classic minibatch gradient descent optimization (Hinton et al. 2012). To address this, domain-aware minibatch gradient descent is proposed, where the core idea is constructing minibatches using training samples from the same task and the same imaging modality. Practically, the training data is initially divided into seven groups based on their task attributes: report generation, classification, detection, segmentation, VQA, region captioning, and longitudinal comparisons. Subsequently, minibatches for each group are dynamically generated by randomly sampling training data with matching input imaging modalities. This means that each minibatch should consist of homogeneous data pertaining to a specific task and a single imaging modality. For example, one minibatch could comprise exclusively of samples featuring the segmentation task on CT scans, while another may contain only samples with detection annotations on CXRs.

During each training iteration, the process begins by randomly selecting an imaging modality. Subsequently, from the task pool linked to this modality, a task and sample data pertaining to that specific task is randomly chosen. Gradient descent is applied separately for each minibatch. This allows the model to optimize specifically for the task and the imaging types in that batch, leading to more efficient learning and better overall performance. Different loss functions tailored to different task types are also utilized. For example, a cross entropy loss is used for vision-language tasks, while a combination of the cross entropy and regression losses is applied to the detection task. For the segmentation task both the focal loss (Lin et al. 2020) and the DICE loss (Sudre et al. 2017) is employed, with equal weights assigned to each.

### Implementation Details

For the 2D vision encoder in the multi-modal input coordinator, the base version of Swin Transformer (Liu et al. 2021) pretrained on ImageNet (Deng et al. 2009) is used. This encoder is characterized by its four-stage structure, a window size of seven, a patch size of four, and an initial feature dimension of 128. For the 3D vision encode, the encoder architecture from the 3D UNet (Ciçek et al. 2016) is adopted. For specific tasks like report generation, classification, open-ended VQA, and longitudinal study comparisons, the encoder processes the input images through a random cropping technique, where the cropped area ranges from 50% to 100% of the original image. These cropped images are then resized to a standard dimension of 224×224 pixels with three channels. Different augmentation techniques are applied based on the nature of the task. For chest organ and skin lesion segmentation tasks, a random horizontal flip is applied to each image. In the case of abdomen CT scans, a more complex manipulation is performed by flipping each 3D volume over a random axis. To efficiently manage the volume of visual tokens, MedVersa utilizes an adaptive average pooling strategy, standardizing the output length to nine. Additionally, the system implements two distinct linear projectors for 2D and 3D data. Each projector comprises a fully connected layer, transforming each pooled visual token into a 1D vector of 4,096 elements.

The LLM-based orchestrator was initialized using the model weights of Llama-2-Chat (Touvron et al. 2023). The training of the orchestrator in MedVersa employs the Low-Rank Adaptation (LoRA) strategy (Hu et al. 2021), focusing on parameter efficiency. LoRA utilizes the concept of low-rank matrix decomposition to approximate a large weight matrix in neural network layers. By setting the rank and alpha values of LoRA to 16, the method ensures efficient training while modifying only a fraction of the model parameters. The AdamW optimizer (Loshchilov and Hutter 2017), in combination with a cosine learning rate scheduler, is used for optimization. Training parameters are meticulously set, with an initial learning rate of 3e-4 and a minimum of 3e-6, over 500,000 training iterations. The first 3,000 iterations involve a linear warm-up phase, starting with a learning rate of 1e-7. Finally, the training infrastructure comprises 24 NVIDIA A100 GPUs (80G), achieving a global batch size of 96. This setup allows the training stage to be completed within a 72-hour window, demonstrating the system's efficiency and robustness in handling complex multi-modal AI tasks

### Baselines

CisGen. This is a differentiable end-to-end method with three parts: a classifier, a generator, and an interpreter (Nguyen et al. 2021). The classifier learns disease features through context modeling and a disease-state aware mechanism. The generator turns the disease information into a medical report. The interpreter then reviews and refines these reports, ensuring they align with the classifier's findings. The inventors have empirically found ClsGen showed more consistent performance compared to popular report generation approaches, such as R2Gen (Chen et al. 2020) and M2Trans (Miura et al. 2020).

Deep AUC maximization (DAM). DAM optimizes deep learning models by directly maximizing the area under the receiver operating characteristic curve (AUC) (Yuan et al. 2021). This method is particularly relevant for addressing complex classification problems, especially when dealing with imbalanced datasets. The DAM supervised method was included as a baseline for chest pathology classification, which currently is state-of-the-art on the CheXpert dataset (Tiu et al. 2022).

MAIRA-1. This is a specialist large multi-modal model for report generation from Microsoft (Hyland et al. 2023). It adopted the LLaVA-1.5 architecture (Liu et al. 2023; Liu et al. 2023). MAIRA-1 also benefits from the use of GPT-3.5 for data augmentation, adding 131,558 reports with paraphrased findings and indication sections to the training set. MAIRA-1 produces reports with state-of-the-art quality.

Med-PaLM M. This is a large generalist biomedical AI system from Google Tu et al. 2023). Med-PaLM M was built by finetuning with biomedical data on top of PaLM-E Driess et al. 2023), a generalist multi-modal FM trained on non-medical images and text. Here, comparison to its best variant that has 84 billion parameters was made, which maintains the state-of-the-art in the task of report generation.

PTLM. PTLM is the state-of-the-art approach on open-ended medical visual question answering (van Sonsbeek et al. 2023). PTLM maps the extracted visual features to a set of learnable tokens, which can directly prompt the language model for parameter-efficient finetuning.

EKAID. EKAID integrates the expert knowledge graphs into representation learning (Hu et al. 2023). This is an image-difference model that is sensitive to anatomical structures, allowing it to extract image-difference features that are pertinent to the progression of diseases and interventions. EKAID presents state-of-the-art results in the task of longitudinal study comparisons.

MiniGPT-v2. This is a new multi-modal foundation model that can caption bounding boxes on natural images (Chen et al. 2023). Specifically, it accepts box coordinates as inputs and outputs a caption that describes the objects within the box. MiniGPT-v2 was fine-tuned on the region-of-interest captioning task.

CRCKD. This approach aims to bring similar image pairs from the same skin class closer together in both teacher and student models while pushing apart dissimilar image pairs from different skin classes (Xing et al. 2021). It is a widely adopted baseline and shows competitive performance for categorizing skin lesions. Note that both CRCKD and MedVersa were trained directly on the raw, imbalanced HAM10000 dataset.

YOLOv5. YOLOv5 is a state-of-the-art, real-time object detection algorithm that is part of the YOLO (You Only Look Once) family (Jocher et al. 2020). Following its predecessors in providing fast and accurate object detection capabilities, YOLOv5 has been widely used for detecting abnormalities in medical images (Mohiyuddin et al. 2022; Wan et al. 2021; Luo et al. 2021).

nnUNet. nnUNet is a self-configuring method for deep learning-based biomedical image segmentation (Isensee et al. 2021). This framework is versatile in handling various medical imaging datasets, employing different configurations and preprocessing steps depending on the dataset characteristics. For instance, it adapts the network topologies, such as 2D UNet and 3D UNet, according to the specific requirements of medical segmentation tasks.

nnSAM. The nnSAM architecture integrates the robust and effective feature extraction abilities of Segment Anything Model (Kirillov et al. 2023) with the adaptive configuration strengths of nnUNet (Li et al. 2023). This combination maximizes the potential of each model, with SAM providing high-quality feature extraction and nnUNet enabling the system to automatically adjust to the unique demands of each dataset. nnSAM shows state-of-the-art results in the data-efficient segmentation task.

### REFERENCES

1. Rajpurkar, P. et al. CheXNet: Radiologist-Level Pneumonia Detection on Chest X-Rays with Deep Learning. arXiv [cs.CV](2017).
2. Wang, X. et al. ChestX-Ray8: Hospital-scale chest X-ray database and benchmarks on weakly-supervised classification and localization of common thorax diseases. in 2017 IEEE Conference on Computer Vision and Pattern Recognition (CVPR) 2097-2106 (IEEE, 2017).
3. Irvin, J. et al. CheXpert: A Large Chest Radiograph Dataset with Uncertainty Labels and Expert Comparison. AAAI 33, 590-597 (2019).
4. Johnson, A. E. W. et al. MIMIC-CXR, a de-identified publicly available database of chest radiographs with free-text reports. Sci Data 6, 317 (2019).
5. Tiu, E. et al. Expert-level detection of pathologies from unannotated chest X-ray images via self-supervised learning. Nat Biomed Eng 6, 1399-1406 (2022).
6. Liu, Y. et al. A deep learning system for differential diagnosis of skin diseases. Nat. Med. 26, 900-908 (2020).
7. Esteva, A. et al. Dermatologist-level classification of skin cancer with deep neural networks. Nature 542, 115-118 (2017).
8. Daneshjou, R. et al. Disparities in dermatology AI performance on a diverse, curated clinical image set. Sci Adv 8, eabg6147 (2022).
9. Joshi, G. et al. FDA approved Artificial Intelligence and Machine Learning (AI/ML)-Enabled Medical Devices: An updated landscape. bioRxiv (2022) doi:10.1101/2022.12.07.22283216.
10. Moor, M. et al. Foundation models for generalist medical artificial intelligence. Nature 616, 259-265 (2023).
11. Rajpurkar, P. & Lungren, M. P. The Current and Future State of AI Interpretation of Medical Images. N. Engl. J. Med. 388, 1981-1990 (2023).
12. Bommasani, R. et al. On the Opportunities and Risks of Foundation Models. arXiv [cs.LG](2021).
13. Tu, T. et al. Towards Generalist Biomedical AI. arXiv [cs.CL](2023).
14. Wu, C., Zhang, X., Zhang, Y., Wang, Y. & Xie, W. Towards generalist foundation model for radiology by leveraging web-scale 2D&3D medical data. arXiv [cs.CV](2023).
15. Moor, M. et al. Med-Flamingo: a Multimodal Medical Few-shot Learner. arXiv [cs.CV](2023).
16. Lu, M. Y. et al. A Foundational Multimodal Vision Language AI Assistant for Human Pathology. arXiv [cs.CV](2023).
17. Huang, Z., Bianchi, F., Yuksekgonul, M., Montine, T. J. & Zou, J. A visual-language foundation model for pathology image analysis using medical Twitter. Nat. Med. 29, 2307-2316 (2023).
18. Chen, T. et al. A unified sequence interface for vision tasks. arXiv [cs.CV]31333-31346 (2022).
19. Wang, W. et al. VisionLLM: Large Language Model is also an Open-Ended Decoder for Vision-Centric Tasks. arXiv [cs.CV](2023).
20. Zhang, H. et al. GLIPv2: Unifying localization and Vision-language understanding. arXiv [cs.CV]36067-36080 (2022).
21. Hyland, S. L. et al. MAIRA-1: A specialised large multimodal model for radiology report generation. arXiv [cs.CL](2023).
22. Jocher, G., Nishimura, K., Mineeva, T. & Vilariño, R. yolov5. Code repository (2020).
23. Papineni, K., Roukos, S., Ward, T. & Zhu, W.-J. Bleu: a Method for Automatic Evaluation of Machine Translation, in Proceedings of the 40th Annual Meeting of the Association for Computational Linguistics (eds. Isabelle, P., Charniak, E. & Lin, D.) 311-318 (Association for Computational Linguistics, Philadelphia, Pennsylvania, USA, 2002).
24. Zhang, T., Kishore, V., Wu, F., Weinberger, K. Q. & Artzi, Y. BERTScore: Evaluating Text Generation with BERT. arXiv [cs.CL](2019).
25. Smit, A. et al. CheXbert: Combining Automatic Labelers and Expert Annotations for Accurate Radiology Report Labeling Using BERT. arXiv [cs.CL](2020).
26. Jain, S. et al. RadGraph: Extracting Clinical Entities and Relations from Radiology Reports. arXiv [cs.CL](2021).
27. Yu, F. et al. Evaluating progress in automatic chest X-ray radiology report generation. Patterns (N Y) 4, 100802 (2023).
28. Nguyen, H. T. N. et al. Automated Generation of Accurate & Fluent Medical X-ray Reports. arXiv [cs.CL](2021).
29. Demner-Fushman, D. et al. Preparing a collection of radiology examinations for distribution and retrieval. J. Am. Med. Inform. Assoc. 23, 304-310 (2016).
30. Isensee, F., Jaeger, P. F., Kohl, S. A. A., Petersen, J. & Maier-Hein, K. H. nnU-Net: a self-configuring method for deep learning-based biomedical image segmentation. Nat. Methods 18, 203-211 (2021).
31. Li, Y., Jing, B., Li, Z., Wang, J. & Zhang, Y. nnSAM: Plug-and-play Segment Anything Model Improves nnUNet Performance. arXiv [cs.CV](2023).
32. van Sonsbeek, T., Derakhshani, M. M., Najdenkoska, I., Snoek, C. G. M. & Worring, M. Open-Ended Medical Visual Question Answering Through Prefix Tuning of Language Models. arXiv [cs.CV](2023).
33. Yuan, Z., Yan, Y., Sonka, M. & Yang, T. Large-scale robust deep AUC maximization: A new surrogate loss and empirical studies on medical image classification, in 2021 IEEE/CVF International Conference on Computer Vision (ICCV) 3040-3049 (IEEE, 2021).
34. Xing, X. et al. Categorical Relation-Preserving Contrastive Knowledge Distillation for Medical Image Classification, in Medical Image Computing and Computer Assisted Intervention MICCAI 2021 163-173 (Springer International Publishing, 2021).
35. Johnson, A. et al. MIMIC-CXR-JPG—chest radiographs with structured labels. physionet.org https://doi.org/10.13026/8360-t248 (2019).
36. Endo, M., Krishnan, R., Krishna, V., Ng, A. Y. & Rajpurkar, P. Retrieval-Based Chest X-Ray Report Generation Using a Pre-trained Contrastive Language-Image Model. in Proceedings of Machine Learning for Health (eds. Roy, S. et al.) vol. 158 209-219 (PMLR, 2021).
37. Wu, J. T. et al. Chest ImaGenome Dataset for Clinical Reasoning. arXiv [cs.CV](2021).
38. Wu, J. T. et al. Chest ImaGenome dataset for clinical reasoning. arXiv [cs.CV](2021).
39. Hu, X. et al. Expert Knowledge-Aware Image Difference Graph Representation Learning for Difference-Aware Medical Visual Question Answering. in Proceedings of the 29th ACM SIGKDD Conference on Knowledge Discovery and Data Mining 4156-4165 (Association for Computing Machinery, New York, NY, USA, 2023).
40. Tschandl, P., Rosendahl, C. & Kittler, H. The HAM10000 dataset, a large collection of multi-source dermatoscopic images of common pigmented skin lesions. Sci Data 5, 180161 (2018).
41. Codella, N. et al. Skin Lesion Analysis Toward Melanoma Detection 2018: A Challenge Hosted by the International Skin Imaging Collaboration (ISIC). arXiv [cs.CV](2019).
42. Alam, T. M. et al. An Efficient Deep Learning-Based Skin Cancer Classifier for an Imbalanced Dataset. Diagnostics (Basel) 12, (2022).
43. Ma, J. et al. AbdomenCT-1K: Is Abdominal Organ Segmentation a Solved Problem?IEEE Trans. Pattern Anal. Mach. Intell. 44, 6695-6714 (2022).
44. Boecking, B. et al. Making the Most of Text Semantics to Improve Biomedical Vision-Language Processing. in Computer Vision—ECCV 2022 1-21 (Springer Nature Switzerland, 2022).
45. Boecking, B. et al. MS-CXR: Making the most of text semantics to improve biomedical vision-language processing. PhysioNet https://doi.org/10.13026/B90J-VB87 (2022).
46. Zhu, D., Chen, J., Shen, X., Li, X. & Elhoseiny, M. MiniGPT-4: Enhancing Vision-Language Understanding with Advanced Large Language Models. arXiv [cs.CV](2023).
47. Chen, J. et al. MiniGPT-v2: large language model as a unified interface for vision-language multi-task learning. arXiv [cs.CV](2023).
48. Li, C. et al. LLaVA-Med: Training a Large Language-and-Vision Assistant for Biomedicine in One Day. arXiv [cs.CV](2023).
49. Vaswani, A. et al. Attention is all you need. Adv. Neural Inf. Process. Syst. 30, (2017).
50. Ciçgek, O., Abdulkadir, A., Lienkamp, S. S., Brox, T. & Ronneberger, O. 3D U-Net: Learning Dense Volumetric Segmentation from Sparse Annotation, in Medical Image Computing and Computer-Assisted Intervention—MICCAI 2016 424-432 (Springer International Publishing, 2016).
51. He, K., Zhang, X., Ren, S. & Sun, J. Spatial Pyramid Pooling in Deep Convolutional Networks for Visual Recognition. IEEE Trans. Pattern Anal. Mach. Intell. 37, 1904-1916 (2015).
52. Ba, J. L., Kiros, J. R. & Hinton, G. E. Layer Normalization. arXiv [stat.ML](2016).
53. Touvron, H. et al. Llama 2: Open Foundation and Fine-Tuned Chat Models. arXiv [cs.CL](2023).
54. Kudo, T. & Richardson, J. SentencePiece: A simple and language independent subword tokenizer and detokenizer for Neural Text Processing. arXiv [cs.CL](2018).
55. Isensee, F. et al. nnU-Net: Self-adapting Framework for U-Net-Based Medical Image Segmentation. arXiv [cs.CV](2018).
56. He, K., Zhang, X., Ren, S. & Sun, J. Deep residual learning for image recognition. arXiv [cs.CV]770-778 (2015).
57. Deng, J. et al. ImageNet: A large-scale hierarchical image database. in 2009 IEEE Conference on Computer Vision and Pattern Recognition 248-255 (IEEE, 2009).
58. Jaegle, A. et al. Perceiver: General Perception with Iterative Attention. in Proceedings of the 38th International Conference on Machine Learning (eds. Meila, M. & Zhang, T.) vol. 139 4651-4664 (PMLR, 18-24 Jul. 2021).
59. Taori, R. et al. Stanford alpaca: An instruction-following llama model. Preprint at (2023).
60. Wei, J. et al. Finetuned Language Models Are Zero-Shot Learners. arXiv [cs.CL](2021).
61. Chung, H. W. et al. Scaling Instruction-Finetuned Language Models. arXiv [cs.LG](2022).
62. Singhal, K. et al. Large language models encode clinical knowledge. Nature 620, 172-180 (2023).
63. Hinton, G., Srivastava, S. & Swersky, K. Neural Networks for Machine Learning Lecture 6a Overview of mini—batch gradient descent. http://www.cs.toronto.edu/-hinton/coursera/lecture6/lec6.pdf (2012).
64. Lin, T.-Y., Goyal, P., Girshick, R., He, K. & Dollar, P. Focal Loss for Dense Object Detection. IEEE Trans. Pattern Anal. Mach. Intell. 42, 318-327 (2020).
65. Sudre, C. H., Li, W., Vercauteren, T., Ourselin, S. & Jorge Cardoso, M. Generalised Dice Overlap as a Deep Learning Loss Function for Highly Unbalanced Segmentations. Deep Learn Med Image Anal Multimodal Learn Clin Decis Support (2017) 2017, 240-248 (2017).
66. Liu, Z. et al. Swin Transformer: Hierarchical Vision Transformer using Shifted Windows. in 2021 IEEE/CVF International Conference on Computer Vision (ICCV) 9992-10002 (IEEE, 2021).
67. Hu, E. J. et al. LoRA: Low-Rank Adaptation of Large Language Models. arXiv [cs.CL](2021).
68. Loshchilov, I. & Hutter, F. Decoupled Weight Decay Regularization. arXiv [cs.LG](2017).
69. Chen, Z., Song, Y., Chang, T.-H. & Wan, X. Generating Radiology Reports via Memory-driven Transformer. arXiv [cs.CL](2020).
70. Miura, Y., Zhang, Y., Tsai, E. B., Langlotz, C. P. & Jurafsky, D. Improving Factual Completeness and Consistency of Image-to-Text Radiology Report Generation. arXiv [cs.CL](2020).
71. Liu, H., Li, C., Wu, Q. & Lee, Y. J. Visual Instruction Tuning. arXiv [cs.CV](2023).
72. Liu, H., Li, C., Li, Y. & Lee, Y. J. Improved Baselines with Visual Instruction Tuning. arXiv [cs.CV](2023).
73. Driess, D. et al. PaLM-E: An Embodied Multimodal Language Model. arXiv [cs.LG](2023).
74. Mohiyuddin, A. et al. Breast Tumor Detection and Classification in Mammogram Images Using Modified YOLOv5 Network. Comput. Math. Methods Med. 2022, U.S. Pat. No. 1,359,019 (2022).
75. Wan, J., Chen, B. & Yu, Y. Polyp Detection from Colorectum Images by Using Attentive YOLOv5. Diagnostics (Basel) 11, (2021).
76. Luo, Y., Zhang, Y., Sun, X., Dai, H. & Chen, X. Intelligent Solutions in Chest Abnormality Detection Based on YOLOv5 and ResNet50. J. Healthc. Eng. 2021, U.S. Pat. No. 2,267,635 (2021).
77. Kirillov, A. et al. Segment Anything. arXiv [cs.CV](2023).

### Example Embodiments

(1) A method for performing medical tasks using a medical artificial intelligence (MAI) system, the MAI system comprising a trained large language model (LLM) and a plurality of task-specific software tools, the method comprising: using at least one computer hardware processor to perform: (A) receiving multi-modal input comprising image input and a request that the MAI system perform at least one medical task on the multi-modal input; (B) processing at least a portion of the multi-modal input using the trained LLM to obtain LLM output, the LLM output indicating that zero, one, or multiple tasks are to be additionally performed by at least one of the plurality of task-specific software tools; (C) when the LLM output indicates that zero tasks are to be additionally performed, outputting at least some of the LLM output as a response to the request; and (D) when the LLM output indicates that one or multiple tasks are to be additionally performed, processing at least some of the multi-modal input, using the at least one of the plurality of task-specific software tools and the LLM output, to obtain at least one task-specific output; outputting a response generated using the at least some of the LLM output and the at least one task-specific output as a response to the request.

(2) The method of (1), wherein the image input comprises one or more medical images comprising one or more radiographs, dermoscopy images, computed tomography scans, pathology images, ultrasound images, endoscopy images, and/or magnetic resonance imaging (MRI) images.

(3) The method of (1), wherein the image input comprises one or more two-dimensional medical images, one or more three-dimensional images, and/or one or more videos.

(4) The method of (1), wherein the image input comprises a single image.

(5) The method of (1), wherein the image input comprises multiple images.

(6) The method of (5), wherein the multiple images comprise multiple views of at least a portion of patient.

(7) The method of (5), wherein the multiple images comprise a same view of at least a portion of a patient at multiple points in time.

(8) The method of (1), wherein the response comprises a multi-modal output.

(9) The method of (8), wherein the multi-modal output comprises a text output and an image output.

(10) The method of (9), wherein the LLM output comprises the text output and the at least one task-specific output comprises the image output.

(11) The method of (1), wherein the at least one medical task comprises one or more vision-language tasks comprising one or more of medical report generation, longitudinal study comparison, region-of-interest captioning, open-ended visual question answering, and/or abnormality (e.g., skin lesion) classification, and/or one or more vision-centric tasks comprising one or more medical image analysis tasks including one or more of anatomical structure identification, abnormality characterization, chest abnormality detection, lesion segmentation, and/or organ segmentation.

(12) The method of (1), further comprising processing the multi-modal input to obtain a tokenized representation of the image input and the text input at least in part by processing the text input to generate text tokens and processing the image input to generate visual tokens, and wherein the at least a portion of the multi-modal output comprises the tokenized representation of the image input and the text input.

(13) The method of (12), wherein processing the multi-modal input to obtain the tokenized representation of the image input and the text input further comprises adapting the visual tokens into text tokens using a model trained to transform visual tokens into text tokens.

(14) The method of (12), wherein: when the image input comprises a two-dimensional (2D) image, the processing the image input comprises processing the 2D image using a 2D vision encoder; and when the image input comprises a three-dimensional (3D) image, the processing the image input comprises processing the 3D image using a 3D vision encoder.

(15) The method of (1), further comprising: determining whether the LLM output indicates zero, one, or multiple tasks are to be additionally performed by the at least one of the plurality of task-specific software tools.

(16) The method of (15), wherein determining whether the LLM output indicates zero, one, or multiple tasks are to be additionally performed by the at least one of the plurality of task-specific software tools comprises determining whether the LLM output comprises at least one tag associated with at least one respective task.

(17) The method of (16), further comprising identifying the at least one of the plurality of task-specific software tools using the at least one tag, wherein the at least one tag comprises a first tag associated with a first task, and the at least one of the plurality of task-specific software tools is trained to perform the first task.

(18) The method of (1), wherein: the image input comprises a two-dimensional image; the LLM output indicates that a detection task is to be additionally performed by the at least one of the plurality of task-specific software tools; the at least one of the plurality of task-specific software tools comprises a task-specific software tool trained to perform the detection task on the two-dimensional image; the at least one task-specific output comprises a second textual output, wherein obtaining the at least one task-specific output comprises generating the second textual output; and the response comprises the LLM output and the second textual output, wherein outputting the response comprises outputting the LLM output and the second textual output.

(19) The method of (1), wherein: the image input comprises a three-dimensional image; the LLM output indicates that a segmentation task is to be additionally performed by the at least one of the plurality of task-specific software tools; the at least one of the plurality of task-specific software tools comprises a task-specific software tool trained to perform the segmentation task on the three-dimensional image; the at least one task-specific output comprises an image output, wherein obtaining the at least one task-specific output comprises generating the image output; and the response comprises the image output and the LLM output, wherein outputting the response comprises outputting the LLM output and the image output.

(20) The method of (1), wherein: the image input comprises a plurality of two-dimensional images; and the LLM output indicates that zero additional tasks are to be additionally performed by the at least one of the plurality of task-specific software tools.

(21) The method of (1), further comprising indexing latent embeddings of task specification tokens corresponding to the tasks to be additionally performed by the at least one of the plurality of task-specific software tools and encoding the indexed latent embeddings into a repository comprising the plurality of task-specific software tools.

(22) The method of (1), further comprising updating a repository storing the plurality of task-specific software tools, wherein updating the repository comprises adjusting how one or more of the plurality of task-specific software tools performs a task and/or adding one or more additional task-specific software tools to the repository.

(23) The method of (1), wherein the response to the request of the MAI system comprises one or more of a medical report, a segmented image, an indication of a classified medical condition, a comparison of longitudinal study images, an indication of a detected anatomical structure and/or an indication of a detected abnormality.

(24) The method of (1), further comprising training the trained LLM using a plurality of tags, each tag of the plurality of tags is associated a respective task of the tasks to be additionally performed by the at least one of the plurality of task-specific software tools.

(25) The method of (1), further comprising optimizing the LLM using domain-aware minibatch gradient descent.

(26) The method of (25), wherein optimizing the LLM using the domain-aware minibatch gradient descent creates homogeneous minibatches for training, each of the homogenous minibatches specific to a single type of task or tasks to be performed by the orchestrator or at least one of the plurality of task-specific software tools and a single type of imaging modality.

(27) The method of (1), wherein the plurality of task-specific software tools comprise one or more task-specific trained machine learning models and the at least one of the plurality of task-specific software tools comprises at least one task-specific trained machine learning model.

(28) A method for performing medical tasks using a medical artificial intelligence (MAI) system, the MAI system comprising a plurality of modules including a multi-modal input coordinator module, an orchestrator module comprising a trained large language model (LLM), and a plurality of task-specific software tools, the method comprising: executing the multi-modal input coordinator module, using at least one computer hardware processor, to perform: (A) receiving multi-modal input comprising image input and text input indicating a request that the MAI system perform at least one medical task on the multi-modal input; (B) processing the multi-modal input to obtain a tokenized representation of the image input and the text input; and executing the orchestrator module, using the at least one computer hardware processor, to perform: (C) processing the tokenized representation using the trained LLM to obtain LLM output at least partially responsive to the request, the LLM output comprising latent embeddings and textual output, the LLM output indicating zero, one, or multiple tasks are to be additionally performed by at least one of the plurality of task-specific software tools; (D) when the LLM output indicates that zero tasks are to be additionally performed by the at least one of the plurality of task-specific software tools, outputting the textual output as a response to the request of the MAI system; and (E) when the LLM output indicates that one or multiple tasks are to be additionally performed by the at least one of the plurality of task-specific software tools, identifying, based on the LLM output and from among the plurality of task-specific software tools, a first task-specific software tool; generating, from the latent embeddings and the multi-modal input, first input for the first task-specific software tool and processing the first input with the first task-specific software tool to obtain a first task-specific output; generating an integrated response to the request of the MAI system using the textual output produced by the trained LLM and the first task-specific output generated by the first task-specific software tool; and outputting the integrated response as a response to the request of the MAI system.

(29) The method of (28), wherein the image input comprises one or more medical images comprising one or more radiographs, dermoscopy images, and/or computed tomography scans.

(30) The method of (28), wherein the image input comprises one or more two-dimensional medical images, one or more three-dimensional images, and/or one or more videos.

(31) The method of (28), wherein the image input comprises a single image.

(32) The method of (28), wherein the image input comprises multiple images.

(33) The method of (32), wherein the multiple images comprise multiple views of at least a portion of patient.

(34) The method of (32), wherein the multiple images comprise a same view of at least a portion of a patient at multiple points in time.

(35) The method of (28), wherein the integrated output comprises a multi-modal output.

(36) The method of (35), wherein the multi-modal output comprises a text output and an image output.

(37) The method of (36), wherein the textual output of the LLM output comprises the text output and the first task-specific output comprises the image output.

(38) The method of (28), wherein the at least one medical task comprises one or more vision-language tasks comprising one or more of medical report generation, longitudinal study comparison, region-of-interest captioning, open-ended visual question answering, and/or abnormality classification and/or one or more vision-centric tasks comprising one or more medical image analysis tasks including one or more of anatomical structure identification, abnormality characterization, chest abnormality detection, skin lesion segmentation, and/or organ segmentation.

(39) The method of (28), wherein processing the multi-modal input to obtain the tokenized representation of the image input and the text input comprises processing the text input to generate text tokens and processing the image input to generate visual tokens.

(40) The method of (39), wherein processing the multi-modal input to obtain the tokenized representation of the image input and the text input further comprises adapting the visual tokens into text tokens using a model trained to transform visual tokens into text tokens.

(41) The method of (39), wherein: when the image input comprises a two-dimensional (2D) image, the processing the image input comprises processing the 2D image using a 2D vision encoder; and when the image input comprises a three-dimensional (3D) image, the processing the image input comprises processing the 3D image using a 3D vision encoder.

(42) The method of (28), further comprising: determining whether the LLM output indicates zero, one, or multiple tasks are to be additionally performed by the at least one of the plurality of task-specific software tools.

(43) The method of (42), wherein determining whether the LLM output indicates zero, one, or multiple tasks are to be additionally performed by the at least one of the plurality of task-specific software tools comprises determining whether the textual output comprises at least one tag associated with at least one respective task.

(44) The method of (43), wherein the identifying the first task-specific software tool comprises identifying the first task-specific software tool using the at least one tag, wherein the at least one tag comprises a first tag associated with a first task, and the first task-specific software tool is trained to perform the first task.

(45) The method of (28), wherein: the image input comprises a two-dimensional image; the LLM output indicates that a detection task is to be additionally performed by the at least one of the plurality of task-specific software tools; the first-task specific software tool comprises a task-specific software tool trained to perform the detection task on the two-dimensional image; the first task-specific output comprises a second textual output, wherein generating the first task-specific output comprises generating the second textual output; and the integrated response comprises the textual output of the LLM output and the second textual output, wherein outputting the integrated response comprises outputting the textual output of the LLM output and the second textual output.

(46) The method of (28), wherein: the image input comprises a three-dimensional image; the LLM output indicates that a segmentation task is to be additionally performed by the at least one of the plurality of task-specific software tools; the first task-specific software tool comprises a task-specific software tool trained to perform the segmentation task on the three-dimensional image; the first task-specific output comprises an image output, wherein generating the first task-specific output comprises generating the image output; and the integrated response comprises the image output and the textual output of the LLM output, wherein outputting the integrated response comprises outputting the textual output of the LLM output and the image output.

(47) The method of (28), wherein: the image input comprises a plurality of two-dimensional images; and the LLM output indicates that zero additional tasks are to be additionally performed by the at least one of the plurality of task-specific software tools.

(48) The method of (28), further comprising indexing latent embeddings of task specification tokens corresponding to the tasks to be additionally performed by the at least one of the plurality of task-specific software tools and encoding the indexed latent embeddings into a repository comprising the plurality of task-specific software tools.

(49) The method of (28), further comprising updating a repository storing the plurality of task-specific software tools, wherein updating the repository comprises adjusting how one or more of the plurality of task-specific software tools performs a task and/or adding one or more additional task-specific software tools to the repository.

(50) The method of (28), wherein the response to the request of the MAI system comprises one or more of a medical report, a segmented image, an indication of a classified medical condition, a comparison of longitudinal study images, an indication of a detected anatomical structure and/or an indication of a detected abnormality.

(51) The method of (28), further comprising training an LLM, to obtain the trained LLM, by using a plurality of tags, each tag of the plurality of tags is associated a respective task of the tasks to be additionally performed by the at least one of the plurality of task-specific software tools.

(52) The method of (28), further comprising training an LLM, to obtain the trained LLM, using referring image instructions, which enclose the visual tokens of different images with different text identifiers and incorporating the text identifiers into text instructions to help the LLM specify different images.

(53) The method of (28), further comprising: optimizing the plurality of modules using domain-aware minibatch gradient descent.

(54) The method of (53), wherein optimizing the plurality of modules using the domain-aware minibatch gradient descent creates homogeneous minibatches for training, each of the homogenous minibatches specific to a single type of task of tasks to be performed by the orchestrator or at least one of the plurality of task-specific software tools and a single type of imaging modality.

(55) The method of (28), wherein the plurality of task-specific software tools comprise one or more task-specific trained machine learning models and the first task-specific software tool comprises a first task-specific trained machine learning model.

(56) A method for training a medical artificial intelligence (MAI) system to perform medical tasks, the MAI system comprising an LLM and a plurality of task-specific software tools, wherein the LLM is to be trained to process multi-modal input, containing image input and a request that the MAI system perform at least one medical task on the multi-modal input, to obtain corresponding LLM output at least partially responsive to the request and indicating that zero, one, or multiple tasks are to be additionally performed by at least one of the plurality of task-specific software tools, the method comprising: obtaining training data comprising multiple multi-modal inputs, each particular one of the multiple multi-modal inputs comprising a respective image input and a respective request that the MAI system perform at least one respective medical task on the particular multi-modal input; training the LLM using the training data to obtain a trained LLM.

(57) The method of (58), further comprising using the trained LLM according to the method of any one of (1)-(55).

(58) The method of (58), wherein the training is performed using domain-aware minibatch gradient descent.

(59) A system comprising: at least one computer hardware processor; and at least one non-transitory computer-readable storage medium having encoded thereon instructions that, when executed by the at least one computer hardware processor, cause the at least one computer hardware processor to perform the method of any of (1)-(58).

(60) At least one non-transitory computer-readable storage medium having encoded thereon instructions that, when executed by at least one computer hardware processor, cause the at least one computer hardware processor to perform the method of any of (1)-(58).

(61) A medical artificial intelligence system, named Orchestrated Generalist Medical AI (0-GMAI), characterized by: an orchestrator module using a large language model to dynamically integrate multi-modal medical data input, including radiographs, dermoscopy images, and computed tomography (CT) scans, alongside clinical language data, and execute an extensive array of tasks, including but not limited to report generation, classification, visual question answering, detection, and segmentation; a multi-modal input coordinator to adaptively coordinate multi-modal input and use the memory-efficient vision-language adapters with a simplified three-layer design to map the image tokens to language space; and the ability to process and interpret a combination of medical images and clinical language data using image and text tokens, capable of generating diverse outputs such as medical reports, segmented images, classified medical conditions, and detection of anatomical structures and abnormalities.

(62) The system of (61), wherein the orchestrator module is capable of: (a) defining labels for a variety of tasks, including but not limited to vision-language integration, detection, segmentation, classification, and comparative analysis; (b) incorporating task-specific tokens <Task> into the labels for vision-centric tasks and training the large language model using these labeled tasks; (c) independently carrying out vision-language tasks, including but not limited to generating medical reports and answering questions about images; and (d) indexing and encoding latent embeddings of task specification tokens into specialist tools for executing a range of vision-centric tasks, such as pathology detection and organ segmentation.

(63) The system of (61), wherein the coordinator module is capable of: (a) processing 2D and 3D medical images into visual tokens using 2D and 3D vision encoders, respectively; and (b) using the vision-language adapters to map the visual tokens to language space.

(64) The system of (61), further comprising: (a) a repository of specialist visual modeling tools, each designed for specific types of medical image analysis tasks, including anatomical detection, pathology classification, and segmentation of various organs and lesions; and (b) a mechanism for the regular update and maintenance of the repository of specialist tools to ensure adaptability to new diagnostic challenges and imaging technologies.

(65) A method for medical image interpretation using an orchestrated generalist medical AI system, the method comprising: (a) receiving and processing multi-modal medical data through a coordinator module; (b) integrating the processed multi-modal medical data and executing varying tasks through an orchestrator module that employs a large language model to adaptively invoke external specialist visual modeling tools for executing vision-centric tasks; (c) utilizing referring image instructions to specify and differentiate between multiple input images for complex comparative and diagnostic analyses; (d) optimizing different modules using domain-aware minibatch gradient descent; and (e) generating a comprehensive multi-modal output that includes diagnostic information, medical reports, and detailed image analyses derived from the multi-modal medical data.

(66) The method of (65), wherein the operations of receiving and processing multi-modal medical data use the swin transformer as the 2D encoder, the encoder backbone of 3D U-Net as the 3D encoder, the Llama tokenizer as the text tokenizer, and the memory-efficient vision-language adapters wherein the adapters feature an efficient design with just three layers—adaptive pooling, layer normalization, and linear projection—focusing on reducing the number of visual tokens to manage GPU memory costs effectively.

(67) The method of (65), wherein the method for constructing the orchestrator module includes defining the labels for vision-language tasks and vision-centric tasks, training the large language model on these tasks, indexing the latent embeddings of the task specification tokens, and encoding the indexed embeddings into the specialist tool.

(68) The method of (65), wherein the specialist tools include the visual detection tools, the 2D visual segmentation tool, and the 3D visual segmentation tool.

(69) The method of (64), wherein the referring image instructions enclose the visual tokens of different images with different text identifiers and incorporate these identifiers into the text instructions to help the model specify different images.

(70) The method of (65), wherein the domain-aware minibatch gradient descent creates homogeneous minibatches for training, each specific to a single task type and imaging modality, applying domain-aware minibatch gradient descent optimization to enhance the system's capability in generating accurate and diverse outputs.

(71) The method of (65), wherein the multi-modal output includes, but is not limited to, automated generation of medical reports, segmented images, classified medical conditions, detection and analysis of anatomical structures, and comparison of longitudinal study images, all based on the interpretation of medical scans and clinical context.

What is claimed is:

1. A method for performing medical tasks using a medical artificial intelligence (MAI) system, the MAI system comprising a trained large language model (LLM) and a plurality of task-specific software tools, the method comprising:
using at least one computer hardware processor to perform:
(A) receiving multi-modal input comprising image input and a request that the MAI system perform at least one medical task on the multi-modal input;
(B) processing at least a portion of the multi-modal input using the trained LLM to obtain LLM output, the LLM output indicating that zero, one, or multiple tasks are to be additionally performed by at least one of the plurality of task-specific software tools;
(C) when the LLM output indicates that zero tasks are to be additionally performed, outputting at least some of the LLM output as a response to the request; and
(D) when the LLM output indicates that one or multiple tasks are to be additionally performed,
processing at least some of the multi-modal input, using the at least one of the plurality of task-specific software tools and the LLM output, to obtain at least one task-specific output;
outputting a response generated using the at least some of the LLM output and the at least one task-specific output as a response to the request.

2. The method of claim 1, wherein the image input comprises one or more medical images comprising one or more radiographs, dermoscopy images, computed tomography scans, pathology images, ultrasound images, endoscopy images, and/or magnetic resonance imaging (MRI) images.

3. The method of claim 1, wherein the image input comprises one or more two-dimensional medical images, one or more three-dimensional images, and/or one or more videos.

4. The method of claim 1, wherein the image input comprises a single image.

5. The method of claim 1, wherein the image input comprises multiple images.

6. The method of claim 5, wherein the multiple images comprise multiple views of at least a portion of patient.

7. The method of claim 5, wherein the multiple images comprise a same view of at least a portion of a patient at multiple points in time.

8. The method of claim 1, wherein the response comprises a multi-modal output.

9. The method of claim 8, wherein the multi-modal output comprises a text output and an image output.

10. The method of claim 9, wherein the LLM output comprises the text output and the at least one task-specific output comprises the image output.

11. The method of claim 1, wherein the at least one medical task comprises one or more vision-language tasks comprising one or more of medical report generation, longitudinal study comparison, region-of-interest captioning, open-ended visual question answering, and/or abnormality (e.g., skin lesion) classification, and/or one or more vision-centric tasks comprising one or more medical image analysis tasks including one or more of anatomical structure identification, abnormality characterization, chest abnormality detection, lesion segmentation, and/or organ segmentation.

12. The method of claim 1, further comprising processing the multi-modal input to obtain a tokenized representation of the image input and the text input at least in part by processing the text input to generate text tokens and processing the image input to generate visual tokens, and wherein the at least a portion of the multi-modal output comprises the tokenized representation of the image input and the text input.

13. The method of claim 12, wherein processing the multi-modal input to obtain the tokenized representation of the image input and the text input further comprises adapting the visual tokens into text tokens using a model trained to transform visual tokens into text tokens.

14. The method of claim 12, wherein:
when the image input comprises a two-dimensional (2D) image, the processing the image input comprises processing the 2D image using a 2D vision encoder; and
when the image input comprises a three-dimensional (3D) image, the processing the image input comprises processing the 3D image using a 3D vision encoder.

15. The method of claim 1, further comprising:
determining whether the LLM output indicates zero, one, or multiple tasks are to be additionally performed by the at least one of the plurality of task-specific software tools.

16. The method of claim 15, wherein determining whether the LLM output indicates zero, one, or multiple tasks are to be additionally performed by the at least one of the plurality of task-specific software tools comprises determining whether the LLM output comprises at least one tag associated with at least one respective task.

17. The method of claim 16, further comprising identifying the at least one of the plurality of task-specific software tools using the at least one tag, wherein the at least one tag comprises a first tag associated with a first task, and the at least one of the plurality of task-specific software tools is trained to perform the first task.

18. The method of claim 1, wherein:
the image input comprises a two-dimensional image;
the LLM output indicates that a detection task is to be additionally performed by the at least one of the plurality of task-specific software tools;
the at least one of the plurality of task-specific software tools comprises a task-specific software tool trained to perform the detection task on the two-dimensional image;
the at least one task-specific output comprises a second textual output, wherein obtaining the at least one task-specific output comprises generating the second textual output; and
the response comprises the LLM output and the second textual output, wherein outputting the response comprises outputting the LLM output and the second textual output.

19. A method for performing medical tasks using a medical artificial intelligence (MAI) system, the MAI system comprising a plurality of modules including a multi-modal input coordinator module, an orchestrator module comprising a trained large language model (LLM), and a plurality of task-specific software tools, the method comprising:
executing the multi-modal input coordinator module, using at least one computer hardware processor, to perform:
(A) receiving multi-modal input comprising image input and text input indicating a request that the MAI system perform at least one medical task on the multi-modal input;
(B) processing the multi-modal input to obtain a tokenized representation of the image input and the text input; and
executing the orchestrator module, using the at least one computer hardware processor, to perform:
(C) processing the tokenized representation using the trained LLM to obtain LLM output at least partially responsive to the request, the LLM output comprising latent embeddings and textual output, the LLM output indicating zero, one, or multiple tasks are to be additionally performed by at least one of the plurality of task-specific software tools;
(D) when the LLM output indicates that zero tasks are to be additionally performed by the at least one of the plurality of task-specific software tools, outputting the textual output as a response to the request of the MAI system; and
(E) when the LLM output indicates that one or multiple tasks are to be additionally performed by the at least one of the plurality of task-specific software tools,
identifying, based on the LLM output and from among the plurality of task-specific software tools, a first task-specific software tool;
generating, from the latent embeddings and the multi-modal input, first input for the first task-specific software tool and processing the first input with the first task-specific software tool to obtain a first task-specific output;
generating an integrated response to the request of the MAI system using the textual output produced by the trained LLM and the first task-specific output generated by the first task-specific software tool; and
outputting the integrated response as a response to the request of the MAI system.

20. A system comprising:
at least one computer hardware processor; and
at least one non-transitory computer-readable storage medium having encoded thereon instructions that, when executed by the at least one computer hardware processor, cause the at least one computer hardware processor to perform a method for performing medical tasks using a medical artificial intelligence (MAI) system, the MAI system comprising a trained large language model (LLM) and a plurality of task-specific software tools, the method comprising:
using the at least one computer hardware processor to perform:
(A) receiving multi-modal input comprising image input and a request that the MAI system perform at least one medical task on the multi-modal input;
(B) processing at least a portion of the multi-modal input using the trained LLM to obtain LLM output, the LLM output indicating that zero, one, or multiple tasks are to be additionally performed by at least one of the plurality of task-specific software tools;
(C) when the LLM output indicates that zero tasks are to be additionally performed, outputting at least some of the LLM output as a response to the request; and
(D) when the LLM output indicates that one or multiple tasks are to be additionally performed,
processing at least some of the multi-modal input, using the at least one of the plurality of task-specific software tools and the LLM output, to obtain at least one task-specific output;
outputting a response generated using the at least some of the LLM output and the at least one task-specific output as a response to the request.
